SEQUENCE LISTING (3 sequences)

SEQ ID NO: 1 - protein, 149 aa, Staphylococcus aureus
ATSTKKLHKEPATLIKAIDGDTVKLMYKGQPMTFRLLLVDTPETKHPKKGVEKYGPEASAFTKKMVENAKKIEVEFDKGQRTDKYGRGLAYIYADGKMVNEALVRQGLAKVAYVYKPNNTHEQHLRKSEAQAKKEKLNIWSEDNADSGQ

SEQ ID NO: 2 - protein, 143 aa, Staphylococcus aureus
ATSTKKLHKEPATLIKAIDGNTVKLMYKGQPMVFRLLLVDIPEFNEKYGPEAAAFTKKMVENAKKIEVEFDKGQRTDKYGRGLAYIYADGKMVNEALVRQGLAKVAYVYKGNNTHEQLLRKAEAQAKKEKLNIWSEDNADSGQ

SEQ ID NO: 3 - protein, 143 aa, Staphylococcus aureus
ATSTKKLHKEPATLIKAIDGDTVKLMYKGQPMTFRLLLVDTPEFNEKYGPEASAFTKKMVENAKKIEVEFDKGQRTDKYGRGLAYIYADGKMVNEALVRQGLAKVAYVYKGNNTHEQLLRKAEAQAKKEKLNIWSEDNADSGQ

(19) United States
(12) Patent Application Publication
(10) Pub. No.: US 2015/0072360 A1
(43) Pub. Date: Mar. 12, 2015

(54) BIOMARKERS OF PULMONARY HYPERTENSION

(71) Applicant: THE JOHNS HOPKINS UNIVERSITY, BALTIMORE, MD (US)

(72) Inventors: Allen Dale Everett, Glenwood, MD (US); Zongming Fu, Laurel, MD (US)

(73) Assignee: THE JOHNS HOPKINS UNIVERSITY, BALTIMORE, MD (US)

(21) Appl. No.: 14/365,174

(22) PCT Filed: Dec. 14, 2012
(86) PCT No.: PCT/US2012/069895

§ 371 Date: Jun. 13, 2014

Related U.S. Application Data
(60) Provisional application No. 61/570,347, filed on Dec. 14, 2011.

(51) Int. Cl. G01N 30/72; H01J 49/00; G01N 33/543

(52) U.S. Cl. CPC G01N 30/7233; G01N 33/54306; H01J 49/0027; G01N 2800/12; G01N 2800/60; G01N 2800/50; G01N 2333/4727; G01N 2333/4704; G01N 2500/00
USPC 435/7.92; 250/282

(57) ABSTRACT

Methods are disclosed for predicting or diagnosing pulmonary artery hypertension (PAH) and for determining the efficacy of PAH therapy using biomarkers.

### CROSS-REFERENCE TO RELATED APPLICATIONS

This application claims the benefit of U.S. Provisional Application No. 61/570,347, filed Dec. 14, 2011, which is incorporated herein by reference in its entirety.

### FEDERALLY SPONSORED RESEARCH OR DEVELOPMENT

This invention was made with government support under HL099786 awarded by the National Institutes of Health (NIH). The government has certain rights in the invention.

### BACKGROUND

Pulmonary artery hypertension (PAH) in children or adults is a progressive and fatal disease characterized by sustained elevations of pulmonary artery pressure. Associated pulmonary artery hypertension (APAH) is PAH, which is associated with an underlying pulmonary, cardiac, or systemic disease. Idiopathic pulmonary arterial hypertension (IPAH) is a form of PAH that is present in the absence of an identifiable cause or associated underlying disease. Although advances in therapy and survival with PAH have been made, the etiology is still largely unknown.

In terms of diagnostic or prognostic methods for PAH, clinical functional assessment, such as the 6-minute walk, a test that measures the distance that a patient can walk on a flat, hard surface in a period of 6 minutes, is not applicable to children. In addition, specific diagnostic or prognostic biomarkers are lacking.

Vasodilators are the mainstay of PAH therapy. 20-30% of patients, however, do not respond to vasodilators. Non-responders have a poor prognosis and eventually require lung transplantation. Because the pathobiology is unknown, vasodilator therapy has significant morbidity and cost (˜$50,000/year). Further, the diagnostic or prognostic methods to easily and accurately identify patients that are unresponsive to therapy are lacking.

### SUMMARY

In some aspects, the presently disclosed subject matter provides methods for predicting or diagnosing pulmonary artery hypertension (PAH) or determining if the therapy used to treat PAH is effective.

In some aspects, the presently disclosed subject matter provides a method for predicting or diagnosing pulmonary artery hypertension (PAH) in a subject having PAH, at risk of having PAH, or suspected of having PAH, the method comprising (a) obtaining a sample from a subject at risk of having or suspected of having PAH; (b) detecting a level of expression of at least one biomarker in the sample, wherein the at least one biomarker is selected from the group consisting of Protein S100-A8, Protein S100-A9, Alpha-1B-glycoprotein (A1BG), Beta-2-microglobulin (B2M), Calponin-1 (CNN1), Carbonic anhydrase (CA3), (Complement C4-A (C4A), Tenascin-X (TNXB), Pulmonary surfactant-associated protein C (SFTPC), Uteroglobin (SCGB1A1), Periostin (POSTN), Apolipoprotein A-II (APOA2), Collagen alpha-1(XIV) chain (COL14A1), Complement C3 (C3), Apolipoprotein A-1 (APOA1), Antithrombin-III (SERPINC1), von Willebrand factor (VWF), High mobility group protein B1 (HMGB1), Flavin reductase (NADPH) (BLVRB), Fibulin-1 (FBLN1), Heat shock protein beta-6 (HSPB6), BTB/POZ domain-containing protein (KCTD12), Zyxin (ZYX), Carbonic anhydrase 1 (CA1), Alcohol dehydrogenase 1B (ADH1B), Fibulin-5 (FBLN5), Neutrophil gelatinase-associated lipocalin (LCN2), SERPIN H1 (SERPINH1), Periaxin (PRX), Protein S100-A12 (S100A12), Myeloblastin (PRTN3), Alpha-2-macroglobulin (A2M), Serotransferrin (TF), Histone H2B type 1 (HIST1H2BK), Isoform 2 of collagen alpha-1(XVIII) chain (COL18A1), Basement membrane-specific heparin sulfate proteoglycan core protein (HSPG2), Fibrillin-1 (FBN1), Bone marrow stromal antigen 2 (BST2), Matrix metalloproteinase-9 (MMP9), Periplakin (PPL), Serum amyloid A-1 (SAA1), Thrombospondin-1 (THBS1), Tubulin-specific chaperone A (TBCA), Serine-tRNA ligase, cytoplasmic (SARS), and Aldose reductase (AKR1B1); (c) comparing the levels of the at least one biomarker in the sample to the levels of the at least one biomarker in a control sample from a subject or subjects that do not have PAH, and wherein a significant difference between the levels of the at least one biomarker in the sample and the levels of the at least one biomarker in the control sample is indicative that the subject has or is susceptible to developing PAH.

In other aspects, the presently disclosed subject matter provides a method for predicting or diagnosing hypoxia, hypoxic pulmonary artery hypertension (PAH), normoxia PAH, and no hypoxia or PAH in a subject having hypoxia and/or PAH, at risk of having hypoxia and/or PAH, or suspected of having hypoxia and/or PAH, the method comprising: (a) obtaining a sample from a subject at risk of having or suspected of having hypoxia and/or PAH; (b) detecting a level of expression of at least one biomarker in the sample, wherein the at least one biomarker is selected from the group consisting of Mucin-16 (MUC16), Collagen alpha-1(II) chain (COL2A1), Complement factor H (CFH), Complement C1q tumor necrosis factor-related protein 3 (C1QTNF3), Pantetheinase (VNN1), Complement component C8 beta chain (C8B), Collagen alpha-2(I) chain (COL1A2), Histone H2B type 1-K (HIST1H2BK), Plasminogen (PLG), Phospholipid transfer protein (PLTP), Lactotransferrin (LTF), Vimentin (VIM), Histone H4 (HIST1H4A), Apolipoprotein A-IV (APOA4), Multimerin-1 (MMRN1), Clusterin (CLU), Apolipoprotein C-III (APOC3), Vitronectin (VTN), Endothelial cell-specific molecule 1 (ESM1), SPARC (SPARC), Sushi repeat-containing protein (SRPX), Lumican (LUM), Cation-independent mannose-6-phosphate receptor (IGF2R), Coagulation factor V (F5), Periostin (POSTN), and Pentraxin-related protein PTX3 (PTX3); and (c) comparing the levels of the at least one biomarker in the sample to the levels of the at least one biomarker in a corresponding control sample, wherein a significant difference between the levels of the at least one biomarker in the sample and the levels of the at least one biomarker in the control sample is indicative that the subject has or is susceptible to developing hypoxia and/or PAH.

In further aspects, the presently disclosed subject matter provides a method for predicting or diagnosing pulmonary artery hypertension (PAH) in a subject that has or is susceptible to developing PAH by detecting phosphorylation differences on a protein, the method comprising: (a) obtaining a sample from a subject at risk of having PAH; (b) detecting one or more phosphorylation sites on at least one protein in the sample selected from the group consisting of Aquaporin 1, 60S acidic ribosomal protein P2, 60S acidic ribosomal protein PO, Caveolin-1, Epidermal growth factor receptor substrate 15, Cdc42 effector protein 4, and CLIP-associating protein 2; and (c) comparing the phosphorylation sites of the at least one protein in the sample to the phosphorylation sites of the at least one protein in a control sample from a subject or subjects that do not have PAH, wherein a phosphorylation difference between at least one protein in the sample and the at least one protein in the control sample is indicative that the subject has or is susceptible to developing PAH.

In still further aspects, the presently disclosed subject matter provides a method for determining the efficacy of vasodilator therapy in a subject undergoing treatment thereof, the method comprising: (a) obtaining a sample from the subject undergoing vasodilator therapy; (b) detecting a level of expression of at least one biomarker in the sample, wherein the at least one biomarker is selected from the group consisting of Protein S100-A8, Protein S100-A9, Protein S100-A7, Polyubiquitin-B, Protein S100-A12, Plasma kallikrein, Lymphatic vessel endothelial hyaluronic acid receptor 1, Gamma-glutamyl hydrolase, Tetranectin, Bone-derived growth factor, Platelet basic protein, Insulin-like growth factor-binding protein 3, Pigment epithelium-derived factor, Protein S100-A11 calcium binding protein, Prostaglandin-H2 D-isomerase, Transforming growth factor-beta-induced protein, Vasorin, Kallistatin, Osteopontin, L-selectin, Hepatocyte growth factor activator, and Proliferation-inducing protein 33; and (c) comparing the levels of the at least one biomarker in the sample from the subject undergoing vasodilator therapy to the levels of the at least one biomarker in a previous sample from the subject, wherein a significant difference in the levels of the at least one biomarker in the sample from the subject undergoing vasodilator therapy as compared to the levels of the at least one biomarker in the previous sample is indicative that the vasodilator therapy is effective.

In some aspects, the presently disclosed subject matter provides a method for screening for a new PAH therapy, the method comprising: (a) administering a new therapy to a subject known to have PAH; (b) obtaining a sample from the subject; (c) detecting a level of expression of at least one biomarker in the sample, wherein the at least one biomarker is selected from the group consisting of Protein S100-A8, Protein S100-A9, Alpha-1B-glycoprotein (A1 BG), Beta-2-microglobulin (B2M), Calponin-1 (CNN1), Carbonic anhydrase (CA3), (Complement C4-A (C4A), Tenascin-X (TNXB), Pulmonary surfactant-associated protein C (SFTPC), Uteroglobin (SCGB1A1), Periostin (POSTN), Apolipoprotein A-II (APOA2), Collagen alpha-1(XIV) chain (COL14A1), Complement C3 (C3), Apolipoprotein A-1 (APOA1), Antithrombin-III (SERPINC1), von Willebrand factor (VWF), High mobility group protein B1 (HMGB1), Flavin reductase (NADPH) (BLVRB), Fibulin-1 (FBLN1), Heat shock protein beta-6 (HSPB6), BTB/POZ domain-containing protein (KCTD12), Zyxin (ZYX), Carbonic anhydrase 1 (CA1), Alcohol dehydrogenase 1B (ADH1B), Fibulin-5 (FBLN5), Neutrophil gelatinase-associated lipocalin (LCN2), Serpin H1 (SERPINH1), Periaxin (PRX), Protein S100-A12 (S100A12), Myeloblastin (PRTN3), Alpha-2-macroglobulin (A2M), Serotransferrin (TF), Histone H2B type 1 (HIST1H2BK), Isoform 2 of collagen alpha-1(XVIII) chain (COL18A1), Basement membrane-specific heparin sulfate proteoglycan core protein (HSPG2), Fibrillin-1 (FBN1), Bone marrow stromal antigen 2 (BST2), Matrix metalloproteinase-9 (MMP9), Periplakin (PPL), Serum amyloid A-1 (SAA1), Thrombospondin-1 (THBS1), Tubulin-specific chaperone A (TBCA), Serine-tRNA ligase, cytoplasmic (SARS), and Aldose reductase (AKR1B1); (d) comparing the levels of the at least one biomarker in a sample from a subject known to have PAH to the levels of the at least one biomarker in a control sample from a subject or subjects that do not have PAH or to a previous sample from the subject administered the new therapy; and wherein a significant difference between the levels of the at least one biomarker in the sample and levels of the at least one biomarker in the control sample or the previous sample from the subject administered the new therapy is indicative that the new PAH therapy is effective.

In other aspects, the methods of the presently disclosed subject matter comprise detecting the level of expression of at least one biomarker by using a mass spectrometry method. In a particular embodiment, the mass spectrometry method comprises selected reaction monitoring (SRM) or multiple reaction monitoring (MRM).

In still other aspects, the methods of the presently disclosed subject matter further comprise methods of treatment. In further aspects, the methods of treatment comprise informing the patient or a treating physician of the susceptibility of the patient to PAH. In still further aspects, the methods of treatment further comprise a step of administering a therapeutically effective amount of a vasodilator to the subject having PAH.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1 shows one embodiment of the overall proteomic scheme for abundant protein depletion, high performance liquid chromatography (HPLC) separation and mass spectrometry (MS) identification of candidate therapeutic biomarkers in idiopathic pulmonary artery hypertension (IPAH);

FIG. 2 shows levels of circulating Protein S100-A8 (A8), Protein S100-A9 (A9) and osteopontin (OPN) in plasma pediatric pulmonary hypertension responders vs. non-responders; and

FIG. 3 shows the validation of uteroglobulin as a biomarker of pulmonary artery hypertension in patients with PAH.

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

Pulmonary artery hypertension (PAH) in children or adults is a progressive and fatal disease characterized by sustained elevations of pulmonary artery pressure. Diagnostic or prognostic methods to easily and accurately identify patients who are at risk for PAH are lacking. In addition, diagnostic or prognostic methods to identify patients unresponsive to vasodilator therapy, the main therapy used for PAH patients, or other drug therapies, are also lacking.

Accordingly, in some embodiments, the presently disclosed subject matter provides methods for predicting or diagnosing PAH in a subject at risk of having PAH. In other embodiments, methods are provided to identify patients that are unresponsive or responsive to vasodilator therapy. The presently disclosed methods provide a way to intervene before overt PAH occurs, decrease therapeutic morbidity, and appropriately titrate therapy.

The diagnostic markers, prognostic markers, or therapeutic efficacy markers of the presently disclosed methods can be used for the in vivo assessment of pulmonary hypertension in a patient. For example, the presently disclosed methods can be used in critically ill patients where standard non-invasive echocardiograpy imaging and Doppler assessment are non-diagnostic and/or not quantifiable.

In addition, the presently disclosed methods can serve as the basis to evaluate existing and new therapies. For example, the presently disclosed methods can be used to screen and compare treatment protocols or therapeutic drugs and their combinations. The presently disclosed methods also may be directly applied to cell culture or animal models of pulmonary hypertension as a research tool.

The methods of the presently disclosed subject matter comprise the detection of specific biomarkers with changes in levels of expression in subjects having PAH. In some embodiments, at least one biomarker disclosed herein and found in the lung or circulating in patients suspected of having PAH can be measured and compared to controls to determine whether the patient is likely to get or already has PAH. The biomarker may be used alone or in combination with other disclosed biomarkers. It is expected that in some embodiments, combinations of disclosed biomarker panels will improve sensitivity and/or specificity of the methods.

The presently disclosed subject matter allows in vivo assessment of pulmonary hypertension of all etiologies and in patients of all ages. In some embodiments, the methods allow a diagnosis of a range or extent of PAH in a patient. The patient may be an infant, a child, or an adult. In some embodiments, the patient is already presenting symptoms of PAH (overt). In other embodiments, the patient does not show any signs of having or likely to develop PAH (subclinical). It is expected that earlier diagnosis and intervention in a patient, such as an infant or small child, will result in improved outcomes. In further embodiments, the methods provided allow the assessment and monitoring of the efficacy of pulmonary hypertension therapies in infants, children, and adults.

The presently disclosed methods may be used for any type of PAH, such as idiopathic, newborn, and pulmonary hypertension from other causes, such as structural heart disease, lung disease, inflammatory disease, and heart failure. In some embodiments, the type of PAH is selected from the group consisting of idiopathic pulmonary artery hypertension (IPAH), associated pulmonary artery hypertension (APAH), PAH caused by structural heart disease, PAH caused by lung disease, PAH caused by inflammatory disease, PAH caused by heart failure, PAH caused by congenital heart disease, and pulmonary hypertension in the newborn.

The levels of biomarkers observed using the presently disclosed methods are significantly different in a patient or subject having PAH, at risk of having PAH, or suspected of having PAH as compared to the levels of biomarker found in a control subject not having PAH. In some embodiments, the levels of biomarker found in a subject having PAH, at risk of having PAH, or suspected of having PAH are higher than the levels in a control subject, and in other embodiments, the levels are lower. The biomarkers may be found anywhere in the body of a patient, such as the lung, plasma, serum, blood, lymph, saliva and urine. In some embodiments, the sample is selected from the group consisting of lung tissue, blood, plasma, saliva, urine, and serum. In other embodiments, a significant difference means at least a 1.5 fold difference between the levels of at least one biomarker in the sample and the levels of at least one biomarker in the control sample.

Any methods available in the art for identifying or detecting the presently disclosed biomarkers are encompassed herein. For example, the overexpression or underexpression of a biomarker can be detected on a nucleic acid level or a protein level. Nucleic acid-based techniques for assessing expression are well known in the art and include, for example, determining the level of biomarker mRNA in a sample. Isolated mRNA can be used in hybridization or amplification assays that include, but are not limited to, Southern or Northern analyses, polymerase chain reaction analyses and probe arrays.

In some embodiments, the levels of protein are detected. In particular embodiments, the biomarkers of the presently disclosed subject matter can be detected and/or measured by immunoassay. Immunoassays require biospecific capture reagents, such as antibodies to capture the biomarker. In some embodiments, the immunoassay comprises an antibody. Many antibodies are available commercially and in addition, antibodies also can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well-known in the art.

The presently disclosed subject matter includes immunoassays, including but not limited to, sandwich immunoassays including ELISA or fluorescence-based immunoassays, immunoblots, Western blots, immunoprecipitation, immunofluorescence, and immunocytochemistry. Nephelometry is an assay performed in liquid phase, in which antibodies are in solution. Binding of an antigen to an antibody results in changes in absorbance, which is then measured. In a SELDI-based immunoassay, a biospecific capture reagent for the biomarker is attached to the surface of an MS probe, such as a pre-activated protein chip array. The biomarker is then specifically captured on the biochip through this reagent, and the captured biomarker is detected by mass spectrometry. In addition to antibodies, the presently disclosed subject matter includes any other suitable agent (e.g., a peptide, an aptamer, or a small organic molecule) that specifically binds a disclosed biomarker.

In particular embodiments, the levels of protein are detected by by mass spectroscopy, a method that employs a mass spectrometer. Examples of mass spectrometers include time-of-flight, magnetic sector, quadrupole filter, ion trap, ion cyclotron resonance, electrostatic sector analyzer, hybrids or combinations of the foregoing, and the like. In other embodiments, the mass spectrometric method comprises matrix assisted laser desorption/ionization time-of-flight (MALDI-TOF MS or MALDI-TOF). In some other embodiments, the method comprises MALDI-TOF tandem mass spectrometry (MALDI-TOF MS/MS). In yet another embodiment, mass spectrometry can be combined with another appropriate method(s) as may be contemplated by one of ordinatry skill in the art. For example, MALDI-TOF can be combined with trypsin digestion and tandem mass spectrometry as described herein. In particular embodiments, the mass spectrometric method comprises selected reaction monitoring (SRM) or multiple reaction monitoring (MRM), which are highly specific and sensitive mass spectrometry techniques that can selectively quantify compounds within complex mixtures.

In particular embodiments, detecting the level of expression of at least one biomarker occurs by using a mass spectrometry method or an immunoassay method. However, the biomarkers of the presently disclosed subject matter can be detected by other suitable methods. These methods include, but are not limited to, optical methods, biochips, electrochemical methods (voltametry and amperometry techniques), atomic force microscopy, and radio frequency methods, e.g., multipolar resonance spectroscopy. Optical methods include but are not limited to microscopy (both confocal and non-confocal), detection of fluorescence, luminescence, chemiluminescence, absorbance, reflectance, transmittance, and birefringence or refractive index (e.g., surface Plasmon resonance, ellipsometry, a resonant mirror method, a grating coupler waveguide method or interferometry). Biochips generally comprise solid substrates and have a generally planar surface, to which a capture reagent (also called an adsorbent or affinity reagent) is attached. Protein biochips are biochips adapted for the capture of polypeptides. Many protein biochips are described in the art. Another detection method includes an electrochemicaluminescent assay, which uses labels that emit light when electrochemically stimulated.

In some embodiments, a combination of biomarkers is detected. By “combination” it is meant that at least two biomarkers of the presently disclosed subject matter are detected and at least two biomarker levels of expression are compared to the levels of biomarker in a control sample. Accordingly, at least 2, 3, 4, 5, 6, 7, 8, 9, 10 or more biomarkers may be used in a panel of biomarkers in the methods of the presently disclosed subject matter. In some cases, a more accurate determination of PAH can be made by using more than one biomarker.

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. A ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of people who test positive that are actually positive. Negative predictive value is the percentage of people who test negative that are actually negative. Diagnostic tests that use these biomarkers may show a ROC of at least about 0.6, at least about 0.7, at least about 0.8, or at least about 0.9.

The biomarkers are differentially present in subjects with PAH and subjects without PAH, and therefore, are useful in aiding in the determination of whether a subject has or is at risk of having PAH. In certain embodiments, the biomarkers are measured in a patient sample using the methods described herein and compared, for example, to predefined biomarker levels and correlated to PAH status. In particular embodiments, the measurement(s) may then be compared with a relevant diagnostic amount(s), cut-off(s), or multivariate model scores that distinguish a positive PAH status from a negative PAH status. The diagnostic amount(s) represents a measured amount of a biomarker(s) above which or below which a patient is classified as having a particular PAH status. For example, if the biomarker(s) is/are up-regulated compared to normal levels in a patient that has PAH, then a measured amount above the diagnostic cut-off(s) provides a diagnosis of PAH. Alternatively, if the biomarker(s) is/are down-regulated compared to normal levels in a patient that has PAH, then a measured amount at or below the diagnostic cut-off(s) provides a diagnosis of PAH. As is well understood in the art, by adjusting the particular diagnostic cut-off(s) used in an assay, one can increase the sensitivity or specificity of the diagnostic assay. In particular embodiments, the diagnostic cut-off can be determined, for example, by measuring the amount of biomarkers in a statistically significant number of samples from patients with the different PAH statuses, and drawing the cut-off to suit the desired levels of specificity and sensitivity. In some embodiments, a positive result is assumed if a sample is positive for at least one of the biomarkers of the presently disclosed subject matter.

Furthermore, in some embodiments, the values measured for markers of a biomarker panel are mathematically combined and the combined value is correlated to the underlying diagnostic question. Biomarker values may be combined by any appropriate state of the art mathematical method. In a specific embodiment, the presently disclosed subject matter provides methods for determining the risk of developing PAH in a patient. Biomarker percentages, amounts, or patterns are characteristic of various risk states, such as high, medium, or low. The risk of developing PAH is determined by measuring the relevant biomarkers and then either submitting them to a classification algorithm or comparing them with a control or reference amount or sample, i.e., a predefined level or pattern of biomarkers that is associated with the particular risk level.

In some embodiments, the methods comprise a method of treatment for PAH. These methods include informing the patient or a treating physician of the susceptibility of the patient to PAH. In other embodiments, the methods further comprise informing the patient or a treating physician of the susceptibility of the patient to PAH and/or hypoxia. In still other embodiments, the patient is undergoing vasodilator therapy and the methods further comprise informing the patient or treating physician of the effectiveness of the vasodilator therapy. The treating physician is meant to refer to a physician who diagnoses and/or monitors the patient. The physician may be a general practitioner or a physician who specializes in diseases or disorders related to PAH.

Based on the results of the presently disclosed subject matter, further procedures may be indicated, including additional diagnostic tests or therapeutic procedures. Accordingly, in another embodiment, the methods of the presently disclosed subject matter further comprise a step of treating a subject having PAH. In a particular embodiment, the step of treating a subject having PAH comprises administering a therapeutically effective amount of a vasodilator to the subject having PAH.

### I. METHODS FOR PREDICTING OR DIAGNOSING PULMONARY ARTERY HYPERTENSION

A. Methods for Predicting or Diagnosing Pulmonary Artery Hypertension by Detection of Biomarker Expression Levels

In some embodiments, the presently disclosed subject matter provides a method for predicting or diagnosing pulmonary artery hypertension (PAH) in a subject having PAH, at risk of having PAH, or suspected of having PAH, the method comprising: (a) obtaining a sample from a subject having PAH, at risk of having PAH, or suspected of having PAH; (b) detecting a level of expression of at least one biomarker in the sample, wherein the at least one biomarker is selected from the group consisting of Protein S100-A8, Protein S100-A9, Alpha-1B-glycoprotein (A1BG), Beta-2-microglobulin (B2M), Calponin-1 (CNN1), Carbonic anhydrase (CA3), (Complement C4-A (C4A), Tenascin-X (TNXB), Pulmonary surfactant-associated protein C (SFTPC), Uteroglobin (SCGB1A1), Periostin (POSTN), Apolipoprotein A-II (APOA2), Collagen alpha-1(XIV) chain (COL14A1), Complement C3 (C3), Apolipoprotein A-1 (APOA1), Antithrombin-III (SERPINC1), von Willebrand factor (VWF), High mobility group protein B1 (HMGB1), Flavin reductase (NADPH) (BLVRB), Fibulin-1 (FBLN1), Heat shock protein beta-6 (HSPB6), BTB/POZ domain-containing protein (KCTD12), Zyxin (ZYX), Carbonic anhydrase 1 (CA1), Alcohol dehydrogenase 1B (ADH1B), Fibulin-5 (FBLN5), Neutrophil gelatinase-associated lipocalin (LCN2), SERPIN H1 (SERPINH1), Periaxin (PRX), Protein S100-A12 (S100A12), Myeloblastin (PRTN3), Alpha-2-macroglobulin (A2M), Serotransferrin (TF), Histone H2B type 1 (HIST1H2BK), Isoform 2 of collagen alpha-1(XVIII) chain (COL18A1), Basement membrane-specific heparin sulfate proteoglycan core protein (HSPG2), Fibrillin-1 (FBN1), Bone marrow stromal antigen 2 (BST2), Matrix metalloproteinase-9 (MMP9), Periplakin (PPL), Serum amyloid A-1 (SAA1), Thrombospondin-1 (THBS1), Tubulin-specific chaperone A (TBCA), Serine-tRNA ligase, cytoplasmic (SARS), and Aldose reductase (AKR1B1); (c) comparing the levels of the at least one biomarker to the levels of the at least one biomarker in a control sample from a subject or subjects that do not have PAH, and wherein a significant difference between the levels of the at least one biomarker in the sample and the levels of the at least one biomarker in the control sample is indicative that the subject has PAH or is susceptible to developing PAH. In other embodiments, the biomarkers described herein are biomarkers that are found in lung and/or lung pulmonary artery endothelial cells.

In further embodiments, a method is provided wherein at least one biomarker is selected from the group consisting of Protein S100-A8, Protein S100-A9, and uteroglobulin. In other embodiments, a combination of at least two biomarkers in the sample is detected. In further embodiments, a combination of Protein S100-A8, Protein S100-A9, and uteroglobulin is detected.

B. Methods for Predicting Hypoxia and/or Pulmonary Artery Hypertension by Detection of Biomarker Expression Levels

Generalized hypoxia is a condition in which the body as a whole is deprived of adequate oxygen supply as compared to normoxia or normal oxygen levels. Chronic general hypoxia is one of the most frequent inducers of chronic pulmonary hypertension. Hypoxic pulmonary hypertension is a life-threatening condition if left untreated.

Examples shown herein below disclose different patterns of particular biomarker levels in subjects with hypoxic IPAH, hypoxia without IPAH, normoxia with IPAH, and normoxia with no IPAH (a subject that does not have PAH or hypoxia). These patterns can be used as biomarkers to identify whether a subject has hypoxia, IPAH, or both conditions.

Accordingly, the presently disclosed subject matter provides methods to predict or diagnose whether a subject has hypoxic PAH, normoxic PAH, hypoxia only, or no hypoxia or PAH by comparing a level of expression of at least one disclosed biomarker in the subject to a level of the same biomarker(s) in control samples from a subject with hypoxic PAH, a subject with normoxia PAH, a subject with hypoxia and no PAH, and/or a subject with no PAH or hypoxia.

In some embodiments, the presently disclosed subject matter provides a method for predicting or diagnosing hypoxia, hypoxic pulmonary artery hypertension (PAH), normoxia PAH, and no hypoxia or PAH in a subject having hypoxia and/or PAH, at risk of having hypoxia and/or PAH, or suspected of having PAH, the method comprising: (a) obtaining a sample from a subject at risk of having hypoxia and/or PAH; (b) detecting a level of expression of at least one biomarker in the sample, wherein the at least one biomarker is selected from the group consisting of Mucin-16 (MUC16), Collagen alpha-1(II) chain (COL2A1), Complement factor H (CFH), Complement C1q tumor necrosis factor-related protein 3 (C1QTNF3), Pantetheinase (VNN1), Complement component C8 beta chain (C8B), Collagen alpha-2(I) chain (COL1A2), Histone H2B type 1-K (HIST1H2BK), Plasminogen (PLG), Phospholipid transfer protein (PLTP), Lactotransferrin (LTF), Vimentin (VIM), Histone H4 (HIST1H4A), Apolipoprotein A-IV (APOA4), Multimerin-1 (MMRN1), Clusterin (CLU), Apolipoprotein C-III (APOC3), Vitronectin (VTN), Endothelial cell-specific molecule 1 (ESM1), SPARC (SPARC), Sushi repeat-containing protein (SRPX), Lumican (LUM), Cation-independent mannose-6-phosphate receptor (IGF2R), Coagulation factor V (F5), Periostin (POSTN), and Pentraxin-related protein PTX3 (PTX3); and (c) comparing the levels of the at least one biomarker in the sample to the levels of the at least one biomarker in a corresponding control sample, wherein a significant difference between the levels of the at least one biomarker in the sample and the levels of the at least one biomarker in the control sample is indicative that the subject has or is susceptible to developing hypoxia and/or PAH.

In other embodiments, at least one biomarker in the sample is selected from the group consisting of Mucin-16 (MUC16), Collagen alpha-1(II) chain (COL2A1), and Complement factor H (CFH), and the levels of the at least one biomarker change in the subject when the subject is likely to get or has PAH as compared to the levels of biomarker in a subject or subjects that do not have PAH.

In further embodiments, at least one biomarker in the sample is selected from the group consisting of Complement C1q tumor necrosis factor-related protein 3 (C1QTNF3), Pantetheinase (VNN1), Complement component C8 beta chain (C8B), Collagen alpha-2(I) chain (COL1A2), Histone H2B type 1-K (HIST1H2BK), Plasminogen (PLG), Phospholipid transfer protein (PLTP), and Lactotransferrin (LTF), wherein the levels of the at least one biomarker change in the subject when the subject is likely to get or has PAH without hypoxia as compared to the levels of biomarker in a subject that does not have PAH or hypoxia, and wherein no significant difference is seen if the subject has hypoxic PAH as compared to a subject that has only hypoxia and no PAH.

In still further embodiments, at least one biomarker in the sample is selected from the group consisting of Vimentin (VIM), Histone H4 (HIST1H4A), Apolipoprotein A-IV (APOA4), Multimerin-1 (MMRN1), Clusterin (CLU), and Apolipoprotein C-III (APOC3), wherein the levels of the at least one biomarker change in the subject when the subject is likely to get hypoxic PAH as compared to the levels of biomarker in a subject that only has hypoxia, and wherein no significant difference is seen if the subject has normoxic PAH as compared to a subject that has normoxia and no PAH.

In some other embodiments, at least one biomarker in the sample is selected from the group consisting of Vitronectin (VTN), Endothelial cell-specific molecule 1 (ESM1), and SPARC, and the levels of the at least one biomarker increase in the subject when the subject is likely to get hypoxic PAH as compared to the levels of biomarker in a subject that only has hypoxia and decrease in the subject when the subject is likely to get normoxic PAH as compared to the levels of biomarker in a subject that has no hypoxia or PAH.

In further embodiments, at least one biomarker in the sample is selected from the group consisting of Sushi repeat-containing protein (SRPX), Lumican (LUM), Cation-independent mannose-6-phosphate receptor (IGF2R), and Coagulation factor V (F5), and the levels of the at least one biomarker decrease in the subject when the subject is likely to get hypoxic PAH as compared to the levels of biomarker in a subject that only has hypoxia and increase in the subject when the subject is likely to get normoxic PAH as compared to the levels of biomarker in a subject that has no hypoxia or PAH.

C. Methods for Predicting Pulmonary Artery Hypertension by Detection of Protein Phosphorylation Differences

Protein phosphorylation is a post-translational modification of a protein in which an amino acid residue is phosphorylated by a protein kinase by the addition of a covalently bound phosphate group. It has been found herein that particular proteins show phosphorylation differences in subjects with PAH as compared to subjects without PAH. Therefore, these phosphorylation differences can be used to predict or diagnose patients with PAH.

Accordingly, the presently disclosed subject matter provides a method for predicting or diagnosing pulmonary artery hypertension (PAH) in a subject having PAH, at risk of having PAH, or suspected of having PAH by detecting phosphorylation differences on a protein, the method comprising: (a) obtaining a sample from a subject at risk of having PAH; (b) detecting one or more phosphorylation sites on at least one protein in the sample selected from the group consisting of Aquaporin 1, 60S acidic ribosomal protein P2, 60S acidic ribosomal protein PO, Caveolin-1, Epidermal growth factor receptor substrate 15, Cdc42 effector protein 4, and CLIP-associating protein 2; and (c) comparing the phosphorylation sites of the at least one protein in the sample to the phosphorylation sites of the at least one protein in a control sample, wherein a phosphorylation difference between at least one protein in the sample and the at least one protein in the control sample is indicative that the subject has PAH or is susceptible to developing PAH.

Phosphorylated residues on a protein can be detected in a number of ways, such as by phosph-specific antibodies, two-dimensional gel electrophoresis, mass spectrometry, and other standard protocols known to those in the art.

### II. METHODS TO DETERMINE THE EFFICACY OF PULMONARY ARTERY HYPERTENSION THERAPY

As an example of PAH therapy, vasodilator therapy is used in treating PAH. Examples of vasodilator therapy drugs include, but are not limited to, phospho diesterase 5 (PDE5) inhibitors, prostacyclins, and endothelin receptor antagonists, >20-30% of patients, however, do not respond to vasodilators. Non-responders have a poor prognosis and eventually require lung transplantation. In addition, vasodilator therapy has significant morbidity and cost. No easy and accurate PAH specific way to determine if vasodilator therapy is working in a subject of all ages with PAH is currently available.

Accordingly, the presently disclosed subject matter provides methods to monitor or determine if vasodilator therapy is effective on a subject. In some embodiments, methods are disclosed for determining the efficacy of vasodilator therapy in a subject in need thereof, the method comprising: (a) obtaining a sample from the subject undergoing vasodilator therapy; (b) detecting a level of expression of at least one biomarker in the sample, wherein the at least one biomarker is selected from the group consisting of Protein S100-A8, Protein S100-A9, Protein S100-A7, Polyubiquitin-B, Protein S100-A12, Plasma kallikrein, Lymphatic vessel endothelial hyaluronic acid receptor 1, Gamma-glutamyl hydrolase, Tetranectin, Bone-derived growth factor, Platelet basic protein, Insulin-like growth factor-binding protein 3, Pigment epithelium-derived factor, Protein S100-A11 calcium binding protein, Prostaglandin-H2 D-isomerase, Transforming growth factor-beta-induced protein, Vasorin, Kallistatin, Osteopontin, L-selectin, Hepatocyte growth factor activator, and Proliferation-inducing protein 33; and (c) comparing the levels of the at least one biomarker in the sample from the subject undergoing vasodilator therapy to the levels of the at least one biomarker in a previous sample from the subject, wherein a significant difference in the levels of the at least one biomarker in the sample from the subject undergoing vasodilator therapy as compared to the levels of the at least one biomarker in the previous sample is indicative that the vasodilator therapy is effective.

The change in levels of biomarker may be an increase or decrease, depending on the biomarker(s) being assayed. For example, the Protein S100-A8 and Protein S100-A9 biomarkers show a decrease in samples from subjects with PAH as compared to samples from subjects that do not have PAH. In some embodiments, at least one biomarker is selected from the group consisting of Protein S100-A8 and Protein S100-A9. In other embodiments, a combination of at least two biomarkers in the sample is detected. In still other embodiments, a combination of Protein S100-A8 and Protein S100-A9 is detected.

In another embodiment, the method for determining the efficacy of vasodilator therapy in a subject undergoing treatment thereof comprises detecting a level of expression of at least one biomarker selected from the group consisting of Protein S100-A8, Protein S100-A9, Alpha-1B-glycoprotein (A1BG), Beta-2-microglobulin (B2M), Calponin-1 (CNN1), Carbonic anhydrase (CA3), (Complement C4-A (C4A), Tenascin-X (TNXB), Pulmonary surfactant-associated protein C (SFTPC), Uteroglobin (SCGB1A1), Periostin (POSTN), Apolipoprotein A-II (APOA2), Collagen alpha-1(XIV) chain (COL14A1), Complement C3 (C3), Apolipoprotein A-1 (APOA1), Antithrombin-III (SERPINC1), von Willebrand factor (VWF), High mobility group protein B1 (HMGB1), Flavin reductase (NADPH) (BLVRB), Fibulin-1 (FBLN1), Heat shock protein beta-6 (HSPB6), BTB/POZ domain-containing protein (KCTD12), Zyxin (ZYX), Carbonic anhydrase 1 (CA1), Alcohol dehydrogenase 1B (ADH1B), Fibulin-5 (FBLN5), Neutrophil gelatinase-associated lipocalin (LCN2), SERPIN H1 (SERPINH1), Periaxin (PRX), Protein S100-A12 (S100A12), Myeloblastin (PRTN3), Alpha-2-macroglobulin (A2M), Serotransferrin (TF), Histone H2B type 1 (HIST1H2BK), Isoform 2 of collagen alpha-1(XVIII) chain (COL18A1), Basement membrane-specific heparin sulfate proteoglycan core protein (HSPG2), Fibrillin-1 (FBN1), Bone marrow stromal antigen 2 (BST2), Matrix metalloproteinase-9 (MMP9), Periplakin (PPL), Serum amyloid A-1 (SAA1), Thrombospondin-1 (THBS1), Tubulin-specific chaperone A (TBCA), Serine-tRNA ligase, cytoplasmic (SARS), and Aldose reductase (AKR1B1).

In still another embodiment, the method for determining the efficacy of vasodilator therapy in a subject undergoing treatment thereof comprises detecting a level of expression of at least one biomarker selected from the group consisting of Aquaporin 1, 60S acidic ribosomal protein P2, 60S acidic ribosomal protein PO, Caveolin-1, Epidermal growth factor receptor substrate 15, Cdc42 effector protein 4, and CLIP-associating protein 2.

### III. METHODS FOR SCREENING FOR NEW THERAPIES FOR PULMONARY ARTERY HYPERTENSION

The presently disclosed methods can be used to evaluate existing and new therapies in vitro, in vivo, or ex vivo. In some embodiments, the methods can be used to screen drugs in cell culture. For example, a cell can be contacted with a potential therapeutic drug and at least one biomarker disclosed herein can be assayed for levels of expression. As another example, PAH can be monitored or researched in an animal model by using the biomarkers disclosed in the methods described herein. In some embodiments, the methods can be used to screen for new protocols or drugs in a subject by monitoring the biomarkers disclosed herein.

Accordingly, the presently disclosed subject matter provides a method for screening for a new PAH therapy, the method comprising: (a) administering a new therapy to a subject known to have PAH; (b) obtaining a sample from the subject; (c) detecting a level of expression of at least one biomarker in the sample, wherein the at least one biomarker is selected from the group consisting of Protein S100-A8, Protein S100-A9, Alpha-1B-glycoprotein (A1BG), Beta-2-microglobulin (B2M), Calponin-1 (CNN1), Carbonic anhydrase (CA3), (Complement C4-A (C4A), Tenascin-X (TNXB), Pulmonary surfactant-associated protein C (SFTPC), Uteroglobin (SCGB1A1), Periostin (POSTN), Apolipoprotein A-II (APOA2), Collagen alpha-1(XIV) chain (COL14A1), Complement C3 (C3), Apolipoprotein A-1 (APOA1), Antithrombin-III (SERPINC1), von Willebrand factor (VWF), High mobility group protein B1 (HMGB1), Flavin reductase (NADPH) (BLVRB), Fibulin-1 (FBLN1), Heat shock protein beta-6 (HSPB6), BTB/POZ domain-containing protein (KCTD12), Zyxin (ZYX), Carbonic anhydrase 1 (CA1), Alcohol dehydrogenase 1B (ADH1B), Fibulin-5 (FBLN5), Neutrophil gelatinase-associated lipocalin (LCN2), Serpin H1 (SERPINH1), Periaxin (PRX), Protein S100-A12 (S100A12), Myeloblastin (PRTN3), Alpha-2-macroglobulin (A2M), Serotransferrin (TF), Histone H2B type 1 (HIST1H2BK), Isoform 2 of collagen alpha-1(XVIII) chain (COL18A1), Basement membrane-specific heparin sulfate proteoglycan core protein (HSPG2), Fibrillin-1 (FBN1), Bone marrow stromal antigen 2 (BST2), Matrix metalloproteinase-9 (MMP9), Periplakin (PPL), Serum amyloid A-1 (SAA1), Thrombospondin-1 (THBS1), Tubulin-specific chaperone A (TBCA), Serine-tRNA ligase, cytoplasmic (SARS), and Aldose reductase (AKR1B1); (d) comparing the levels of the at least one biomarker to the levels of the at least one biomarker in a control sample from a subject or subjects that do not have PAH or to a previous sample from the subject administered the new therapy, and wherein a significant difference between the levels of the at least one biomarker in the sample and levels of the at least one biomarker in the control sample or the previous sample from the subject administered the new therapy is indicative that the new PAH therapy is effective.

In other embodiments, the new therapy is a drug. In still other embodiments, the subject is a human or an animal.

### IV. KITS FOR THE DETECTION OF PAH BIOMARKERS

The presently disclosed subject matter also relates to kits for practicing the methods of the invention. By “kit” is intended any article of manufacture (e.g., a package or a container) comprising a substrate for collecting a biological sample from the patient and means for measuring the levels of one or more biomarkers as described herein.

Accordingly, in one embodiment, the presently disclosed subject matter provides a diagnostic kit for determining predicting or diagnosing pulmonary artery hypertension (PAH) in a subject having PAH, at risk of having PAH, or suspected of having PAH, the kit comprising: (a) a substrate for collecting a biological sample from the patient; and (b) means for measuring the levels of one or more biomarkers selected from the group consisting of Protein S100-A8, Protein S100-A9, Alpha-1B-glycoprotein (A1BG), Beta-2-microglobulin (B2M), Calponin-1 (CNN1), Carbonic anhydrase (CA3), (Complement C4-A (C4A), Tenascin-X (TNXB), Pulmonary surfactant-associated protein C (SFTPC), Uteroglobin (SCGB1A1), Periostin (POSTN), Apolipoprotein A-II (APOA2), Collagen alpha-1(XIV) chain (COL14A1), Complement C3 (C3), Apolipoprotein A-1 (APOA1), Antithrombin-III (SERPINC1), von Willebrand factor (VWF), High mobility group protein B1 (HMGB1), Flavin reductase (NADPH) (BLVRB), Fibulin-1 (FBLN1), Heat shock protein beta-6 (HSPB6), BTB/POZ domain-containing protein (KCTD12), Zyxin (ZYX), Carbonic anhydrase 1 (CA1), Alcohol dehydrogenase 1B (ADH1B), Fibulin-5 (FBLN5), Neutrophil gelatinase-associated lipocalin (LCN2), Serpin H1 (SERPINH1), Periaxin (PRX), Protein S100-A12 (S100A12), Myeloblastin (PRTN3), Alpha-2-macroglobulin (A2M), Serotransferrin (TF), Histone H2B type 1 (HIST1H2BK), Isoform 2 of collagen alpha-1(XVIII) chain (COL18A1), Basement membrane-specific heparin sulfate proteoglycan core protein (HSPG2), Fibrillin-1 (FBN1), Bone marrow stromal antigen 2 (BST2), Matrix metalloproteinase-9 (MMP9), Periplakin (PPL), Serum amyloid A-1 (SAA1), Thrombospondin-1 (THBS1), Tubulin-specific chaperone A (TBCA), Serine-tRNA ligase, cytoplasmic (SARS), and Aldose reductase (AKR1B1).

In another embodiment, the presently disclosed subject matter provides a diagnostic kit for predicting or diagnosing hypoxia, hypoxic pulmonary artery hypertension (PAH), normoxic PAH, and no hypoxia or PAH in a subject having hypoxia and/or PAH, at risk of having hypoxia and/or PAH, or suspected of having hypoxia and/or PAH, the kit comprising: (a) a substrate for collecting a biological sample from the patient; and (b) means for measuring the levels of one or more biomarkers selected from the group consisting of Mucin-16 (MUC16), Collagen alpha-1(II) chain (COL2A1), Complement factor H (CFH), Complement C1q tumor necrosis factor-related protein 3 (C1QTNF3), Pantetheinase (VNN1), Complement component C8 beta chain (C8B), Collagen alpha-2(I) chain (COL1A2), Histone H2B type 1-K (HIST1H2BK), Plasminogen (PLG), Phospholipid transfer protein (PLTP), Lactotransferrin (LTF), Vimentin (VIM), Histone H4 (HIST1H4A), Apolipoprotein A-IV (APOA4), Multimerin-1 (MMRN1), Clusterin (CLU), Apolipoprotein C-111 (APOC3), Vitronectin (VTN), Endothelial cell-specific molecule 1 (ESM1), SPARC, Sushi repeat-containing protein (SRPX), Lumican (LUM), Cation-independent mannose-6-phosphate receptor (IGF2R), Coagulation factor V (F5), Periostin (POSTN), and Pentraxin-related protein PTX3 (PTX3).

In still another embodiment, the presently disclosed subject matter provides a diagnostic kit for predicting or diagnosing pulmonary artery hypertension (PAH) in a subject having PAH, at risk of having PAH, or suspected of having PAH by detecting phosphorylation differences on a protein, the kit comprising: (a) a substrate for collecting a biological sample from the patient; and (b) means for measuring the levels of one or more biomarkers selected from the group consisting of Aquaporin 1, 60S acidic ribosomal protein P2, 60S acidic ribosomal protein PO, Caveolin-1, Epidermal growth factor receptor substrate 15, Cdc42 effector protein 4, and CLIP-associating protein 2.

In a further embodiment, the presently disclosed subject matter provides a diagnostic kit for determining the efficacy of vasodilator therapy in a subject undergoing thereof, the kit comprising: (a) a substrate for collecting a biological sample from the patient; and (b) means for measuring the levels of one or more biomarkers selected from the group consisting of Protein S100-A8, Protein S100-A9, Protein S100-A7, Polyubiquitin-B, Protein S100-A12, Plasma kallikrein, Lymphatic vessel endothelial hyaluronic acid receptor 1, Gamma-glutamyl hydrolase, Tetranectin, Bone-derived growth factor, Platelet basic protein, Insulin-like growth factor-binding protein 3, Pigment epithelium-derived factor, Protein S100-A11 calcium binding protein, Prostaglandin-H2 D-isomerase, Transforming growth factor-beta-induced protein, Vasorin, Kallistatin, Osteopontin, L-selectin, Hepatocyte growth factor activator, and Proliferation-inducing protein 33.

In more specific embodiments, the kit is provided as an ELISA kit comprising antibodies to at least one biomarker of the presently disclosed subject matter. The ELISA kit may comprise a solid support, such as a chip, microtiter plate (e.g., a 96-well plate), bead, or resin having biomarker capture reagents attached thereon. The kit may further comprise a means for detecting the biomarkers, such as antibodies, and a secondary antibody-signal complex such as horseradish peroxidase (HRP)-conjugated goat anti-rabbit IgG antibody and tetramethyl benzidine (TMB) as a substrate for HRP.

The kit for predicting or diagnosing PAH may be provided as an immunochromatography strip comprising a membrane on which the antibodies are immobilized, and a means for detecting the antibodies, such as gold particle bound antibodies. The types of membranes used are known in the art and include nitrocellulose and PVDF membranes. The kit may also comprise a plastic plate on which a sample application pad, gold particle bound antibodies temporally immobilized on a glass fiber filter, a nitrocellulose membrane on which antibody bands and a secondary antibody band are immobilized and an absorbent pad are positioned in a serial manner, so as to keep continuous capillary flow of blood serum.

In certain embodiments, a patient can be diagnosed by adding blood or blood serum from the patient to the kit and detecting the relevant biomarkers conjugated with antibodies. The method may comprise the steps of collecting blood or blood serum from a patient, separating blood serum from the patient's blood, adding the blood serum from the patient to a diagnostic kit, and detecting the biomarkers conjugated with antibodies. If the biomarkers are present in the sample, the antibodies will bind to the sample, or a portion thereof. In other kit and diagnostic embodiments, blood or blood serum need not be collected from the patient because it is already collected.

In other embodiments, the kit can also comprise a washing solution or instructions for making a washing solution, in which the combination of the capture reagents and the washing solution allows capture of the biomarkers on the solid support for subsequent detection by, for example, antibodies or mass spectrometry. In further embodiments, a kit can comprise instructions in the form of a label or separate insert. For example, the instructions may give information regarding how to collect the sample, how to wash the probe, or the particular biomarkers to be detected, and the like. In yet another embodiment, the kit can comprise one or more containers with biomarker samples that can be used as standard(s) for calibration.

### V. DEFINITIONS

As used herein, the term “comparing” refers to making an assessment of how the proportion, level or cellular localization of one or more biomarkers is a sample from a patient relates to the proportion, level or cellular localization of one or more biomarkers in a control sample. For example, “comparing” may refer to assessing whether the proportion, level or cellular localization of one or more biomarkers in a sample from a patient is the same as, more or less than, or different in proportion, level, or cellular localization of the corresponding one or more biomarkers in a standard or control sample. More particularly, the term may refer to assessing whether the proportion, level, or cellular localization of one or more biomarkers in a patient is the same as, more or less than, different from or otherwise corresponds to the proportion, level, or cellular localization of predefined biomarker levels that correspond to, for example, a patient having PAH, not having PAH, responding to treatment for PAH, not responding to treatment for PAH, likely or not likely to respond to a particular PAH treatment, or having/not having another disease or condition.

As used herein, a “biomarker” is any gene or protein whose level of expression in a cell or tissue is altered in some way compared to that of a normal or healthy cell or tissue. In some embodiments, the amount of biomarker may be changed. In other embodiments, the biomarker may be differentially modified in some way. Biomarkers of the presently disclosed subject matter are selective for PAH. In some cases, proteins are listed as biomarkers but it is understood that the proteins themselves do not need to be detected but nucleic acids correlating to the proteins can be detected instead in the methods of the presently disclosed subject matter.

As used herein, the terms “treat,” treating,” “treatment,” and the like, are meant to decrease, suppress, attenuate, diminish, arrest, the underlying cause of a disease, disorder, or condition, or to stabilize the development or progression of a disease, disorder, condition, and/or symptoms associated therewith. It will be appreciated that, although not precluded, treating a disease, disorder or condition does not require that the disease, disorder, condition or symptoms associated therewith be completely eliminated.

As used herein, the terms “measuring” and “determining” refer to methods which include detecting the level of a biomarker(s) in a sample.

As used herein, the terms “prevent,” “preventing,” “prevention,” “prophylactic treatment” and the like refer to reducing the probability of developing a disease, disorder, or condition in a subject, who does not have, but is at risk of or susceptible to developing a disease, disorder, or condition.

As used herein, the term “subject at risk” of getting a disease refers to estimating that a subject will have a disease or disorder in the future based on the subject's current symptoms, family history, lifestyle choices, and the like.

As used herein, the term “indicative” or “likely” means that the event referred to is probable. For example, if the methods of the presently disclosed subject matter result in a conclusion that the subject is likely to get PAH, that means it is probable that the subject will get PAH.

As used herein, the term “diagnosing” refers to the process of attempting to determine or identify a disease or disorder.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for treating an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

Depending on the specific conditions being treated, the vasodilators may be formulated into liquid or solid dosage forms and administered systemically or locally. The agents may be delivered, for example, in a timed- or sustained-low release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the vasodilators may be formulated and diluted in aqueous solutions, such as in physiologically compatible buffers, such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the vasodilators into dosages suitable for systemic administration is within the scope of the disclosure. With proper choice of carrier and suitable manufacturing practice, the vasodilators may be formulated as solutions and may be administered parenterally, such as by intravenous injection. The vasodilators can be formulated readily using pharmaceutically acceptable carriers well known in the art into dosages suitable for oral administration. The vasodilators can be administered as tablets, pills, capsules, liquids, gels, syrups, slurries, suspensions and the like, for oral ingestion by a subject (e.g., patient) to be treated.

For nasal or inhalation delivery, the vasodilators may be formulated by methods known to those of skill in the art, and may include, for example, but not limited to, examples of solubilizing, diluting, or dispersing substances, such as, saline, preservatives, such as benzyl alcohol, absorption promoters, and fluorocarbons.

In addition to the active ingredients, the vasodilators may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the vasodilators with solid excipients, optionally grinding a resulting mixture, and processing the mixture of granules, after adding suitable auxiliaries, if desired, to obtain tablets or dragee cores. Suitable excipients are, in particular, fillers, such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof, such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler, such as lactose, binders, such as starches, and/or lubricants, such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

As used herein, the term “sample” refers to any sampling of cells, tissues, or bodily fluids in which expression of a biomarker can be detected. The sample may be a part of a subject in vivo or ex vivo. For example, a sample may be blood, serum, plasma, urine, saliva, tissue, lung, lymph or any other part of a subject that can be removed.

As used herein, the term “control sample”, “corresponding control”, or “appropriate control” means any control or standard familiar to one of ordinary skill in the art useful for comparison purposes. For example, the control sample may be taken from a subject or subjects that do not have a specific disease, disorder, or condition, such as PAH and/or hypoxia.

As used herein, the term “level of expression” of a biomarker refers to the amount of biomarker detected. Levels of biomarker can be detected at the transcriptional level, the translational level, and the post-translational level, for example.

As used herein, the terms “significantly different” or “significant difference” mean a level of expression of a biomarker in a sample that is higher or lower than the level of expression of said biomarker in a control sample by at least 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, 3.0 fold, 3.1 fold, 3.2 fold, 3.3 fold, 3.4 fold, 3.5 fold, 3.6 fold, 3.7 fold, 3.8 fold, 3.9 fold, 4.0 fold, 4.1 fold, 4.2 fold, 4.3 fold, 4.4 fold, 4.5 fold, 4.6 fold, 4.7 fold, 4.8 fold, 4.9 fold, 5.0 fold or more.

As used herein, the term “effective” means amelioration of one or more causes or symptoms of a disease or disorder, such as PAH and/or hypoxia. Such amelioration only requires a reduction or alteration, not necessarily elimination, of said causes or symptoms.

As used herein, the term “antibody” is used in the broadest sense and encompasses naturally occurring forms of antibodies and recombinant antibodies such as single-chain antibodies, chimeric and humanized antibodies and multi-specific antibodies as well as fragments an derivatives of all of the foregoing.

As used herein, “hypoxia” refers to an inadequate oxygen supply to the cells and tissues of the body and “normoxia” refers to the condition of having a normal level of oxygen in the body.

Unless defined otherwise, all technical and scientific terms used herein have the meaning commonly understood by a person skilled in the art to which this invention belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Identification of Biomarkers with Different Abundance in Pulmonary Hypertension in Blood that Change in Response to Vasodilator Therapy

The presently disclosed subject matter provides a non-biased, in-depth proteomics approach in patients with pulmonary hypertension. Using longitudinal plasma samples of children with PAH that did or did not respond to drug therapy, significant changes in a number of plasma proteins in patients with PAH were identified.

Plasma prognostic biomarkers were identified by comparing the plasma longitudinally after vasodilator therapy. Using a biomarker development pipeline that culminated with quantitative mass spectroscopy (LTQ Orbitrap Hybrid LC/Mass Spectrometer; Thermo Scientific), longitudinal quantitative expression maps of plasma from children with PAH prior to and after initiation of vasodilator therapy (n=15) were developed.

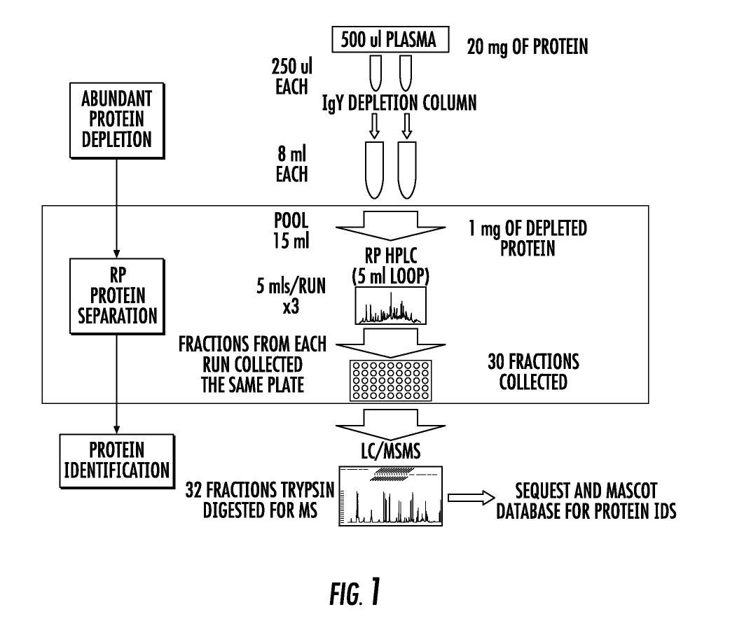

Numerous proteomic methods were compared to determine the best combination to use in exploring the serum proteome. It was found that serum depletion of abundant proteins followed by intact protein separation by 1D HPLC was the best combination while observing a broad range of protein concentrations. All steps in the pipeline were optimized to minimize technical variability. One embodiment of the optimized approach is detailed schematically in FIG. 1.

In this embodiment, plasma was passed through an IgY depletion column to remove abundant proteins. The depleted protein sample was then passed through an HPLC, such as a reversed phase HPLC, which allows separation of proteins even of nearly identical sequences. The proteins in the fractions from the HPLC run were then digested with trypsin in preparation for mass spectrometry. Using this combination of methods allowed the observation of a large number of low abundant cellular and secreted proteins (e.g. skeletal muscle troponin T, nuclear transcription factors, cell adhesion molecules, a cytokine and the stem cell plasma membrane receptor, cKit). Using the LC/MS/MS (liquid chromatography coupled with tandem mass spectrometry) spectra of the tryptic peptides obtained with a LTQ Orbitrap Hybrid LC/Mass Spectrometer (Thermo Scientific), proteins were identified using Sequest and Mascot protein database search engines of the International Protein Index (IPI) human protein database. At least a two peptide coverage was required for confident protein identification. Semiquantitative fold differences were determined using normalized spectral counts of identified proteins. For lung extracts, the pipeline was the same as shown in FIG. 1 except that the IgY depletion step was excluded.

Specifically, LC-MS/MS was performed on a ThermoFisher Easy-nLC 1000 nanoflow LC system coupled on-line to a LTQ OrbiTrap Elite mass spectrometer (ThermoFisher). The peptides were separated by a BioBasic C18 reverse-phase PicoFrit column (300 Å, 5 μm, 75 μm×10 cm, 15 μm tip, New Objective). Peptides were eluted with a 142-min linear gradient from 5 to 45% B (mobile phase A: 2% v/v ACN containing 0.1% v/v formic acid; mobile phase B: 90% v/v ACN containing 0.1% v/v formic acid) at 200 nl/min flow rate. The OrbiTrap was operated with an applied electrospray potential of 3.0 kV and capillary transfer tube temperature of 200° C. in a data-dependent mode where each full MS scan was followed by ten MS/MS scans in which the top 20 most abundant peptide molecular ions detected from the MS scan were dynamically selected for MS/MS analysis using a normalized CID energy of 35%. A dynamic exclusion of 60-s was applied to reduce redundant selection of peptides.

Plasma from children that were undergoing vasodilator therapy that was effective was used to separate and identify the proteins that changed in response to vasodilator therapy (Table 1). Samples were taken from each child at different times during the therapy, including before or just after therapy was initiated and when a child was declared to be improving or worsening. In some embodiments, the proteins that changed over the course of the treatment can be used as biomarkers to determine if a subject is responding to vasodilator therapy.

TABLE 1
Biomarkers in blood that change in response to vasodilator therapy

| | Non-Responders | Responders |
|---|---|---|
| | Mean of | Mean of |
| | Individual | Individual |
| Protein Description | SC Ratio | SC Ratio |
| (With UniProt Accession Nos.) | (Post/Pre) | (Post/Pre) |
| Protein S100-A8 GN = S100A8 | 147.8 | 11.9 |
| {P05109}; S100 calcium binding | | |
| protein A8 (Calgranulin A) | | |
| Polyubiquitin-B GN = UBB | 14.3 | 1.5 |
| {P0CG47} | | |
| Protein S100-A7 GN = S100A7 | 6.3 | 3.3 |
| {P31151} | | |
| Protein S100-A9 GN = S100A9 | 5.2 | 4.4 |
| {P06702}; S100 calcium binding | | |
| protein A9 (Calgranulin B) isoform | | |
| CRA_a OS = Homo sapiens | | |
| GN = S100A9 PE = 2 SV = 1 | | |
| {D3DV36} | | |
| Protein S100-A12 OS = Homo | 3.0 | 20.0 |
| sapiens GN = S100A12 PE = 1 SV = 2 | | |
| {P80511} | | |
| Plasma kallikrein GN = KLKB1 | 2.9 | 1.1 |
| {P03952}; Kallikrein B, plasma | | |
| (Fletcher factor) | | |
| Lymphatic vessel endothelial | 2.5 | 10.7 |
| hyaluronic acid receptor 1 | | |
| GN = LYVE1 {Q9Y5Y7} | | |
| Gamma-glutamyl hydrolase | 2.0 | 1.1 |
| GN = GGH {Q92820} | | |
| Tetranectin GN = CLEC3B | 1.6 | 5.1 |
| {P05452} | | |
| Bone-derived growth factor | 1.5 | 0.8 |
| (Fragment) GN = QSCN6 {Q13876} | | |
| Platelet basic protein GN = PPBP | 1.3 | 5.8 |
| {P02775} | | |
| Insulin-like growth factor-binding | 1.1 | 2.1 |
| protein 3 GN = IGFBP3 {P17936} | | |
| Pigment epithelium-derived factor | 1.1 | 2.1 |
| GN = SERPINF1 {P36955} | | |
| Protein S100-A11 GN = S100A11 | 1.0 | 0.0 |
| {P31949} | | |
| Prostaglandin-H2 D-isomerase | 1.0 | 2.5 |
| GN = PTGDS {P41222} | | |
| Transforming growth factor-beta- | 0.9 | 0.5 |
| induced protein GN = TGFBI | | |
| {Q15582} | | |
| Vasorin GN = VASN {Q6EMK4} | 0.8 | 7.9 |
| Kallistatin OS = Homo sapiens | 0.7 | 3.6 |
| GN = SERPINA4 PE = 1 SV = 3 | | |
| {P29622}; cDNA, FLJ93695, highly | | |
| similar to Homo sapiens serpin | | |
| peptidase inhibitor, clade A (alpha-1 | | |
| antiproteinase, antitrypsin), member | | |
| 4 (SERPINA4), mRNA OS = Homo | | |
| sapiens PE = 2 SV = 1 {B2R815} | | |
| Secreted phosphoprotein 1 | 0.7 | 52.3 |
| (Osteopontin, bone sialoprotein I, | | |
| early T-lymphocyte activation 1), | | |
| isoform CRA_c; GN = SPP1; | | |
| {B2RDA1} | | |
| L-selectin; GN = SELL; {P14151} | 0.5 | 35.7 |
| Hepatocyte growth factor activator; | 0.4 | 1.2 |
| GN = HGFAC; {Q04756} | | |
| Proliferation-inducing protein 33; | 0.3 | 10.3 |
| GN = SPARCL1; {Q8N4S1} | | |

### Example 2

### Protein S100-A8 and Protein S100-A9 Biomarkers are Increased in the Plasma of Patients with Pulmonary Hypertension (PAH)

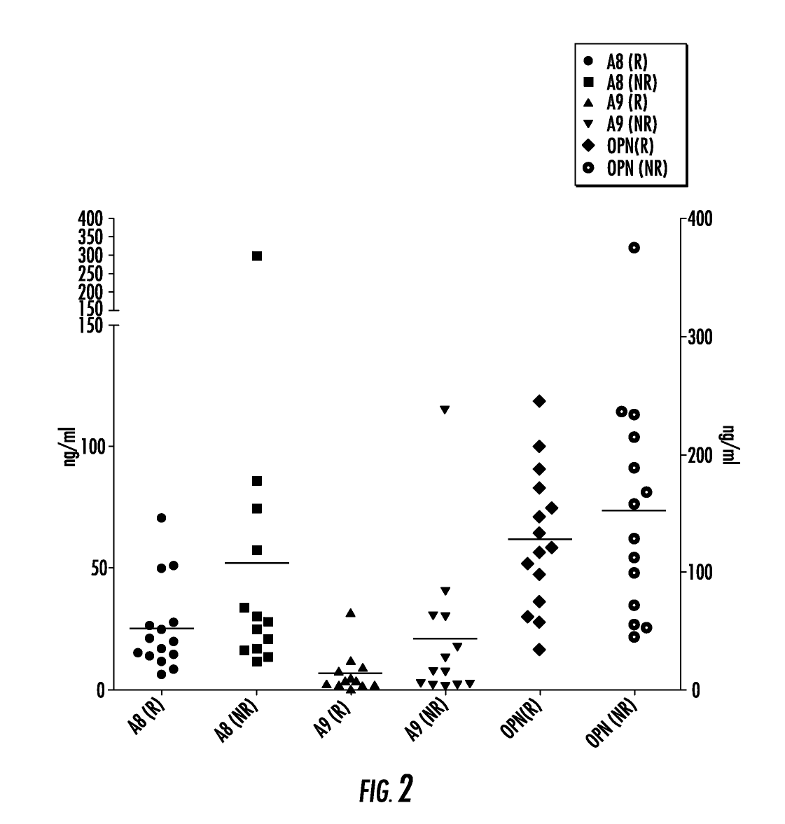

The mass spectrometry data demonstrated that Proteins S100A8 and S100A9 increased in concentration in the plasma of children with pulmonary hypertension that did not respond to vasodilator therapy compared to control samples from children that did respond to vasodilator therapy. Protein S100-A8 and Protein S100-A9 are calcium binding proteins that play a role in inflammation via the RAGE receptor and previously not been associated with PAH. To validate if Protein S100-A8 and Protein S100-A9 were also increased in blood, ELISA assays using commercial kits (R&D Systems) were performed on additional samples taken from children with PAH that had responded or not responded to vasodilator therapy. As shown in FIG. 2, Protein S100-A8 (A8) and Protein S100-A9 (A9) non-responder (NR) levels were both significantly increased in plasma of children as compared to responders (R). Although osteopontin has been shown to be a significant predictor of pulmonary hypertension outcome in adults, in this small group of pediatric patients it was not significant, although the trend was higher in the non-responder group.

In summary, Protein S100-A8 and Protein S100-A9 protein concentrations were significantly increased in plasma and mimic the increase in the plasma identified by mass spectrometry (Table 1). As these two proteins normally circulate as a heterodimer holo-protein, these results confirm the sensitivity and specificity of the mass spectrometry results in identifying PAH biomarker proteins.

### Example 3

### Other Biomarkers Found to be Increased in Lungs and Pulmonary Artery Microvasular Endothelial Cell Lysate of Patients with Pulmonary Hypertension

In this Example, lung tissue and pulmonary artery microvasular endothelial cell lysate from subjects with PAH and lung tissue and pulmonary artery microvasular endothelial cell lysate from subjects without PAH were used for the methods to separate and identify relevant proteins as shown in FIG. 1. Patients with PAH included two groups, acquired PAH (APAH) as a consequence of congenital heart disease and idiopathic PAH (IPAH). Table 2 lists the proteins that were found to have changed levels in the lung in subjects with PAH as compared to subjects without PAH (endo=pulmonary artery microvascular endothelial cells; ec lysate=endothelial cell lysate). Almost all of these proteins, such as uteroglobin, have not been associated with PAH previously. These biomarkers shown in Table 2 can be used independently or in a panel of biomarkers to predict or diagnose a subject that is at risk of PAH.

TABLE 2
Biomarkers found to be increased in lungs and EC
lysate of patients with pulmonary hypertension

| | | | | | | | | Endo |
|---|---|---|---|---|---|---|---|---|
| | | | APAH/ | | IPAH/ | Endo | Endo | IPAH/ |
| Row Labels | Control | APAH | Control | IPAH | Control | control | IPAH | Control |
| Alpha-1B- | | 8.96 | | 4.96 | | | | |
| glycoprotein (A1BG) | | | | | | | | |
| Beta-2-microglobulin | | 5.94 | | 4.95 | | | 1.00 | |
| (B2M) | | | | | | | | |
| Calponin-1 (CNN1) | | 5.00 | | 5.00 | | | | |
| Carbonic anhydrase 3 | | 7.00 | | 9.00 | | | | |
| (CA3) | | | | | | | | |
| Complement C4-A | | 11.00 | | 10.00 | | | | |
| (C4A) | | | | | | | | |
| Tenascin-X (TNXB) | | 3.10 | | 4.09 | | | | |
| Pulmonary | | 3.00 | | 5.00 | | | | |
| surfactant-associated | | | | | | | | |
| protein C (SFTPC) | | | | | | | | |
| Uteroglobin | | 12.00 | | 6.00 | | | | |
| (SCGB1A1) | | | | | | | | |
| Periostin (POSTN) | 5.92 | 56.32 | 9.51 | 44.50 | 7.52 | | | |
| Apolipoprotein A-II | 1.00 | 8.00 | 8.00 | 5.00 | 5.00 | | | |
| (APOA2) | | | | | | | | |
| Collagen alpha- | 2.00 | 15.98 | 7.99 | 28.97 | 14.49 | | | |
| 1(XIV) chain | | | | | | | | |
| (COL14A1) | | | | | | | | |
| Complement C3 (C3) | 12.00 | 59.00 | 4.92 | 48.00 | 4.00 | | | |
| Apolipoprotein A-I | 16.00 | 78.00 | 4.88 | 55.00 | 3.44 | 1.00 | | 0.00 |
| (APOA1) | | | | | | | | |
| Antithrombin-III | 4.00 | 15.00 | 3.75 | 16.00 | 4.00 | | | |
| (SERPINC1) | | | | | | | | |
| von Willebrand factor | 3.00 | 11.00 | 3.67 | 12.00 | 4.00 | 224.00 | 337.00 | 1.50 |
| (VWF) | | | | | | | | |
| High mobility group | 2.97 | 9.89 | 3.33 | 6.93 | 2.33 | 15.98 | 21.97 | 1.37 |
| protein B1 (HMGB1) | | | | | | | | |
| Flavin reductase | 6.00 | 18.00 | 3.00 | 18.00 | 3.00 | 2.00 | 5.00 | 2.50 |
| (NADPH) (BLVRB) | | | | | | | | |
| Fibulin-1 (FBLN1) | 4.00 | 11.00 | 2.75 | 8.00 | 2.00 | | | |
| Heat shock protein | 4.00 | 11.00 | 2.75 | 12.00 | 3.00 | | | |
| beta-6 (HSPB6) | | | | | | | | |
| BTB/POZ domain- | | | | | | | | |
| containing protein | | | | | | | | |
| (KCTD12) | 3.00 | 8.00 | 2.67 | 11.00 | 3.67 | 41.00 | 57.00 | 1.39 |
| Zyxin (ZYX) | 4.92 | 12.80 | 2.60 | 13.79 | 2.80 | 24.60 | 32.52 | 1.32 |
| Carbonic anhydrase 1 | 27.22 | 60.25 | 2.21 | 48.58 | 1.78 | | | |
| (CA1) | | | | | | | | |
| Alcohol | 62.32 | 120.69 | 1.94 | 130.7 | 2.10 | | | |
| dehydrogenase 1B | | | | | | | | |
| (ADH1B) | | | | | | | | |
| Fibulin-5 (FBLN5) | 3.96 | 6.93 | 1.75 | 9.90 | 2.50 | | | |
| Neutrophil | 9.00 | 3.00 | 0.33 | 3.00 | 0.33 | | | |
| gelatinase-associated | | | | | | | | |
| lipocalin (LCN2) | | | | | | | | |
| SERPIN H1 | 8.90 | 2.95 | 0.33 | | 0.00 | 82.49 | 71.67 | 0.87 |
| (SERPIN H1) | | | | | | | | |
| Periaxin (PRX) | 9.00 | 2.00 | 0.22 | 1.00 | 0.11 | | | |
| Protein S100-A12 | 7.00 | 1.00 | 0.14 | 4.00 | 0.57 | | | |
| (S100A12) | | | | | | | | |
| Myeloblastin | 8.00 | 1.00 | 0.13 | 2.00 | 0.25 | | | |
| (PRTN3) | | | | | | | | |
| Alpha-2- | 8.00 | 62.00 | 7.75 | 47.99 | 6.00 | | | |
| macroglobulin (A2M) | | | | | | | | |
| Serotransferrin (TF) | 39.99 | 173.89 | 4.35 | 142.9 | 3.57 | | | |
| Histone H2B type 1 | 16.00 | 52.97 | 3.31 | 37.00 | 2.31 | 110.99 | 122.97 | 1.11 |
| (HIST1H2BK) | | | | | | | | |
| Isoform 2 of Collagen | 4.98 | 15.90 | 3.19 | 14.92 | 3.00 | 17.00 | 22.00 | 1.29 |
| alpha-1(XVIII) chain | | | | | | | | |
| (COL18A1) | | | | | | | | |
| Basement membrane- | 79.00 | 164.00 | 2.08 | 169 | 2.14 | 264.00 | 375.00 | 1.42 |
| specific heparin | | | | | | | | |
| sulfate proteoglycan | | | | | | | | |
| core protein (HSPG2) | | | | | | | | |
| Fibrillin-1 (FBN1) | 11.96 | 5.00 | 0.42 | 4.99 | 0.42 | | | |
| Bone marrow stromal | 3.00 | | | 1.00 | 0.33 | | | |
| antigen 2 (BST2) | | | | | | | | |
| Matrix | 5.00 | | | | | | | |
| metalloproteinase-9 | | | | | | | | |
| (MMP9) | | | | | | | | |
| Periplakin (PPL) | 4.00 | | | 2.00 | 0.50 | | | |
| Serum amyloid A-1 | 10.00 | | | | | | | |
| (SAA1) | | | | | | | | |
| Thrombospondin-1 | 6.00 | | | | | 172.97 | 237.93 | 1.38 |
| (THBS1) | | | | | | | | |
| Tubulin-specific | 3.00 | | | 4.00 | 1.33 | 2.99 | 3.99 | 1.33 |
| chaperone A (TBCA) | | | | | | | | |
| Serine-tRNA ligase, | | | | | | 19.00 | 8.00 | 0.42 |
| cytoplasmic (SARS) | | | | | | | | |
| Aldose reductase | | | | | | 5.99 | 21.98 | 3.67 |
| (AKR1B1) | | | | | | | | |

### Example 4

### Validation of Uteroglobin as a Biomarker of Pulmonary Artery Hypertension

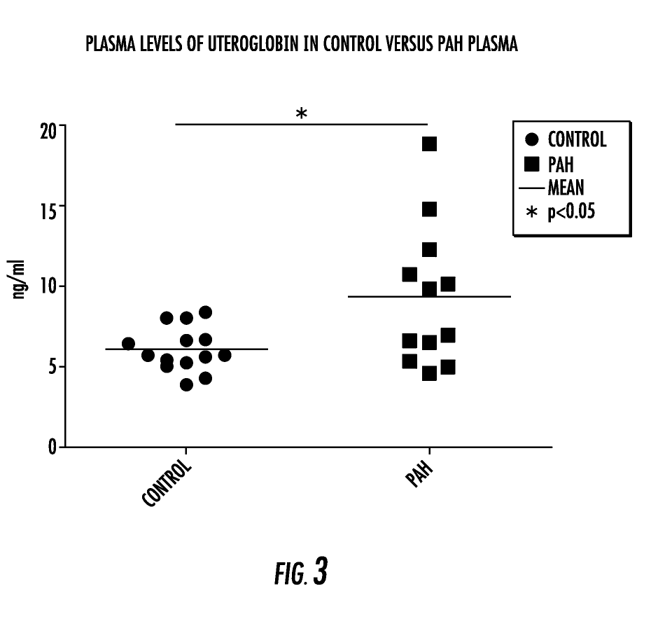

As shown in the mass spectrometry discovery data by spectral counting, uteroglobin, a lung, Clara cell specific protein, was elevated in PAH lungs compared to controls. A commercial ELISA assay for uteroglobin (R&D Systems) was used to validate if elevated uteroglobin in the lung translated to a circulating blood biomarker of PAH. 14 plasma samples matched for age and sex from normal adults presenting for shoulder surgery (presence of lung disease or smoking history were not controlled for) were compared to 12 plasma samples from adults with PAH unresponsive to medical therapy and being evaluated for lung transplant. As shown in FIG. 3, even with this small sample size, circulating uteroglobin was significantly (t test, P=0.014) elevated in the PAH group as compared to controls. This represents the first evidence of a circulating lung specific biomarker for PAH and provides initial validation of the mass spectrometry discoveries as viable biomarkers of PAH.

### Example 5

### Biomarker Levels in Pulmonary Microvascular Endothelial Cells from IPAH and Normal Subjects Exposed to Hypoxia or Normoxia

Chronic general hypoxia is one of the most frequent inducers of chronic pulmonary hypertension. Therefore, subjects can be at risk of getting hypoxia and/or PAH. Examples shown herein below disclose different patterns of particular biomarker expression levels in pulmonary artery microvascular endothelial cells from a subject depending on whether the cells were from a IPAH subject and exposed to hypoxia, hypoxia without IPAH, normoxia with IPAH, and normoxia with no IPAH (a subject that does not have PAH or hypoxia) (Table 3). These patterns can be used as biomarkers to identify whether a subject has hypoxia, IPAH, or both conditions. In these experiments, the “hypoxia control” subjects only had hypoxia and no PAH, the “hypoxia IPAH” subjects had both hypoxia and IPAH, the “normoxia control” subjects had no hypoxia or IPAH, and the “normoxia IPAH” subjects had IPAH without hypoxia.

As can be seen in Table 3, some biomarkers or proteins fell into different groups depending on whether they were differentially expressed under hypoxic or normoxic conditions in cells from subjects with or without PAH as compared to the corresponding controls. Therefore, the presently disclosed subject matter provides methods to predict or diagnose whether a subject has hypoxic PAH, normoxic PAH, hypoxia only, or no hypoxia or PAH by comparing the level of expression of at least one disclosed biomarker in the subject to the level of the same biomarker (s) in control samples from a subject with hypoxic PAH, a subject with normoxia PAH, a subject with hypoxia and no PAH, and/or a subject with no PAH or hypoxia.

TABLE 3
Biomarkers found to be changed under hypoxia or normoxia conditions

| | | | | | | |
|---|---|---|---|---|---|---|
| | Hypoxia | Hypoxia | Hypoxia | Normoxia | Normoxia | Normoxia |
| Protein Description | Control | IPAH | IPAH/CONT | Control | IPAH | IPAH/CONT |

Group 1: Proteins with levels that change significantly in both hypoxia and normoxia subjects

| | | | | | | |
|---|---|---|---|---|---|---|
| Mucin-16 (MUC16) | 1 | 3 | 3 | 1 | 2 | 2 |
| Collagen alpha-1(II) | 2 | | 0 | 2 | 1 | 0.5 |
| chain (COL2A1) | | | | | | |
| Complement factor H | 1 | | 0 | 2 | 1 | 0.5 |
| (CFH) | | | | | | |

Group 2: Proteins with levels that change significantly only in normoxia subjects

| | | | | | | |
|---|---|---|---|---|---|---|
| Complement C1q tumor | | | | 1 | 3 | 3 |
| necrosis factor-related | | | | | | |
| protein 3 (C1QTNF3) | | | | | | |
| Pantetheinase (VNN1) | 6 | 6 | 1 | 3 | 6 | 2 |
| Complement component | | | | 1 | 2 | 2 |
| C8 beta chain (C8B) | | | | | | |
| Collagen alpha-2(I) chain | 1 | 1 | 1 | 2 | 4 | 2 |
| (COL1A2) | | | | | | |
| Histone H2B type 1-K | | | | 1 | 2 | 2 |
| (HIST1H2BK) | | | | | | |
| Plasminogen (PLG) | | | | 1 | 2 | 2 |
| Phospholipid transfer | 2 | 2 | 1 | 2 | 1 | 0.5 |
| protein (PLTP) | | | | | | |
| Lactotransferrin (LTF) | 3 | 2 | 0.67 | 4 | 1 | 0.25 |

Group 3: Proteins with levels that change significantly only in hypoxia subjects

| | | | | | | |
|---|---|---|---|---|---|---|
| Vimentin (VIM) | 1 | 2 | 2 | 2 | 2 | 1 |
| Histone H4 (HIST1H4A) | 2 | 1 | 0.5 | | | |
| Apolipoprotein A-IV | 2 | 1 | 0.5 | 2 | 2 | 1 |
| (APOA4) | | | | | | |
| Multimerin-1 (MMRN1) | 15.98 | 7 | 0.44 | 12.99 | 9.98 | 0.77 |
| Clusterin (CLU) | 6.03 | 2.02 | 0.33 | 3.03 | 2.02 | 0.67 |
| Apolipoprotein C-III | 3 | 1 | 0.33 | 3 | 3 | 1 |
| (APOC3) | | | | | | |

Group 4: Proteins with levels that increase in hypoxia and decrease in normoxia subjects

| | | | | | | |
|---|---|---|---|---|---|---|
| Vitronectin (VTN) | 1 | 3 | 3 | 3 | 1 | 0.33 |
| Endothelial cell-specific | 1 | 2 | 2 | 3 | 1 | 0.33 |
| molecule 1 (ESM1) | | | | | | |
| SPARC (SPARC) | 5.96 | 10.95 | 1.84 | 7.96 | 6.96 | 0.87 |

Group 5: Proteins with levels that decrease in hypoxia and increase in normoxia subjects

| | | | | | | |
|---|---|---|---|---|---|---|
| Sushi repeat-containing | 2 | 1 | 0.5 | 1 | 2 | 2 |
| protein (SRPX) | | | | | | |
| Lumican (LUM) | 4 | 2 | 0.5 | 6 | 7 | 1.17 |
| Cation-independent | 3 | 1 | 0.33 | 3 | 4 | 1.33 |
| mannose-6-phosphate | | | | | | |
| receptor (IGF2R) | | | | | | |
| Coagulation factor V (F5) | 6 | 5 | 0.83 | 3 | 7 | 2.33 |

Other

| | | | | | | |
|---|---|---|---|---|---|---|
| Periostin (POSTN) | 13.95 | 10.98 | 0.79 | 16.96 | 12.98 | 0.77 |
| Pentraxin-related protein | 4 | 3 | 0.75 | 15 | 6 | 0.4 |
| PTX3 (PTX3) | | | | | | |

### Example 6

### Proteins with Phosphorylation Differences in Patients with Pulmonary Hypertension

To determine if phosphorylation differences of proteins in patients with or without pulmonary hypertension could be used to predict or diagnose patients with PAH, lung proteins were digested with trypsin and the phosphorylated peptides were enriched using a titanium dioxide enrichment matrix. The phosphorylated peptides were identified by mass spectrometry using an Orbitrap Elite mass spectrometer (Thermo Scientific). Neutral loss triggered high-energy collisional dissociation (HCD) collision was used to acquire additional MS2.

Proteins with phosphorylation differences in patients with PAH or at risk of getting PAH as compared to control patients are shown in Table 4. The amino acid residues with phosphorylation differences in the proteins are shown as small caps. The number of peptides phosphorylated in each group are shown in the ratios. For example, for the protein aquaporin 1, the protein was found to be phosphorylated 0 times in the IPAH group but it was phosphorylated in 1 out of 4 of the samples in the control group.

Accordingly, the phosphorylation differences in these particular proteins can be used as biomarkers to predict or diagnose patients with PAH.

TABLE 4
Proteins with phosphorylation differences
in pulmonary hypertension

| | | Sequence | IPAH | APAH |
|---|---|---|---|---|
| | | Identi- | vs | vs |
| Protein | | fier for | con- | Con- |
| Name | Peptide | Peptide | trol | trol |
| Aquaporin | (K)VWTSGQVEEYDLDAD | SEQ ID | 0/4 | 1/4 |
| 1 | DINsR(V) | NO: 1 | | |
| 60S acidic | (K)KEESEEsDDDmGFGL | SEQ ID | 12/4 | 4/1 |
| ribosomal | FD(-) | NO: 2 | | |
| protein P2 | (K)KEEsEEsDDDmGFGL | SEQ ID | 17/6 | 20/6 |
| | FD(-) | NO: 3 | | |
| 60S acidic | (K)VEAKEEsEEsDEDmG | SEQ ID | 6/0 | 6/0 |
| ribosomal | FGLFD(-) | NO: 4 | | |
| protein P0 | | | | |
| Caveolin-1 | (K)AmADELsEK(Q) | SEQ ID | 2/5 | 0/5 |
| | | NO: 5 | | |
| Epidermal | (R)SINKLDsPDPFK(L) | SEQ ID | 4/8 | 0/8 |
| growth | | NO: 6 | | |
| factor | | | | |
| receptor | | | | |
| substrate | | | | |
| 15 | | | | |
| Cdc42 | (R)NGAAGPHsPDPLLDE | SEQ ID | 0/0 | 2/0 |
| effector | QAFGDLTDLPVVPK(A) | NO: 7 | | |
| protein 4 | | | | |
| CLIP- | (R)SRsDIDVNAAAGAK | SEQ ID | 4/0 | 0/0 |
| associ- | (A) | NO: 8 | | |
| ating | (R)VLNTGsDVEEAVADA | SEQ ID | 2/0 | 0/0 |
| protein 2 | LK(K) | NO: 9 | | |
| | (R)SRsDIDVNAAAGAK | SEQ ID | 2/0 | 0/0 |
| | (A) | NO: 10 | | |

### Example 7

### Biomarker Amino Acid Sequence Information

Table 5 lists the biomarkers used in the presently disclosed methods. Any of these biomarkers and conservatively modified variants thereof can be used either alone or in combination in the presently disclosed methods to prevent, diagnose, or monitor PAH. In some embodiments, isoforms of the proteins disclosed in Table 5 may also be used in the methods of the presently disclosed subject matter.

TABLE 5
Biomarker amino acid information

| Biomarker Name | NCBI Accession No. |
|---|---|
| Protein S100-A8 | NP_002955 |
| Protein S100-A9 | NP_002956 |
| Protein S100-A7 | NP_002954 |
| Polyubiquitin-B | NP_061828 |
| Protein S100-A12 | NP_005612 |
| Plasma kallikrein | NP_000883 |
| Lymphatic vessel | NP_006682 |
| endothelial hyaluronic acid | |
| receptor 1 | |
| Gamma-glutamyl | NP_003869 |
| hydrolase | |
| Tetranectin | NP_003269 |
| Bone-derived growth | AAA89173 |
| factor | |
| Platelet basic protein | NP_002695 |
| Insulin-like growth factor- | NP_001013416 (Isoform a) |
| binding protein 3 | NP_000589 (Isoform b) |
| Pigment epithelium- | NP_002606 |
| derived factor | |
| Protein S100-A11 calcium | NP_005611 |
| binding protein | |
| Prostaglandin-H2 D- | NP_000945 |
| isomerase | |
| Transforming growth | NP_000349 |
| factor-beta-induced protein | |
| ig-h3 | |
| Vasorin | NP_612449 |
| Kallistatin | NP_006206 |
| Osteopontin | AAA59974 |
| L-selectin | NP_000646 |
| Hepatocyte growth factor | NP_001519 |
| activator | |
| Proliferation-inducing | NP_004675 |
| protein 33 | |
| Alpha-1B-glycoprotein | NP_570602 |
| (A1BG) | |
| Beta-2-microglobulin | NP_004039 |
| (B2M) | |
| Calponin-1 (CNN1) | NP_001290 |
| Carbonic anhydrase 3 | NP_005172 |
| (CA3) | |
| Complement C4-A (C4A) | NP_009224 (Isoform 1) |
| | NP_001239133 (Isoform 2) |
| Tenascin-X (TNXB) | NP_061978 |
| | NP_115859 |
| Pulmonary surfactant- | NP_003009 |
| associated protein C | |
| (SFTPC) | |
| Uteroglobin (SCGB1A1) | NP_003348 |
| Periostin (POSTN) | NP_006466 |
| Apolipoprotein A-II | NP_001634 |
| (APOA2) | |
| Collagen alpha-1(XIV) | Q05707 |
| chain (COL14A1) | |
| Complement C3 (C3) | NP_000055 |
| Apolipoprotein A-I | NP_000030 |
| (APOA1) | |
| Antithrombin-III | NP_000479 |
| (SERPINC1) | |
| von Willebrand factor | NP_000543 |
| (VWF) | |
| High mobility group | NP_002119 |
| protein B1 (HMGB1) | |
| Flavin reductase (NADPH) | NP_000704 |
| (BLVRB) | |
| Fibulin-1 (FBLN1) | NP_006478 |
| Heat shock protein beta-6 | NP_653218 |
| (HSPB6) | |
| BTB/POZ domain- | NP_612453 |
| containing protein | |
| (KCTD12) | |
| Zyxin (ZYX) | NP_001010972 and |
| | NP_003452 |
| Carbonic anhydrase 1 | NP_001122303 |
| (CA1) | |
| Alcohol dehydrogenase 1B | NP_000659 |
| (ADH1B) | |
| Fibulin-5 (FBLN5) | NP_006320 |
| Neutrophil gelatinase- | NP_005555 |
| associated lipocalin | |
| (LCN2) | |
| SERPIN H1 (SERPINH1) | NP_001193943 |
| Periaxin (PRX) | NP_066007 (Isoform 1) |
| | NP_870998 (Isoform 2) |
| Myeloblastin (PRTN3) | NP_002768 |
| Alpha-2-macroglobulin | NP_000005 |
| (A2M) | |
| Serotransferrin (TF) | NP_001054 |
| Histone H2B type 1 | NP_542160 |
| (HIST1H2BK) | |
| Isoform 2 of Collagen | NP_569712 |
| alpha-1(XVIII) chain | |
| (COL18A1) | |
| Basement membrane- | NP_005520 |
| specific heparin sulfate | |
| proteoglycan core protein | |
| (HSPG2) | |
| Fibrillin-1 (FBN1) | NP_000129 |
| Bone marrow stromal | NP_004326 |
| antigen 2 (BST2) | |
| Matrix metalloproteinase-9 | NP_004985 |
| (MMP9) | |
| Periplakin (PPL) | NP_002696 |
| Serum amyloid A-1 | NP_000322 |
| (SAA1) | |
| Thrombospondin-1 | NP_003237 |
| (THBS1) | |
| Tubulin-specific | NP_004598 |
| chaperone A (TBCA) | |
| Serine—tRNA ligase, | NP_006504 |
| cytoplasmic (SARS) | |
| Aldose reductase | NP_001619 |
| (AKR1B1) | |
| Mucin-16 (MUC16) | NP_078966 |
| Collagen alpha-1(II) chain | NP_001835 |
| (COL2A1) | |
| Complement factor H | NP_000177 (Isoform a) |
| (CFH) | NP_001014975 (Isoform b) |
| Complement C1q tumor | NP_112207 (Isoform a) |
| necrosis factor-related | NP_852100 (Isoform b) |
| protein 3 (C1QTNF3) | |
| Pantetheinase (VNN1) | NP_004657 |
| Complement component | NP_000057 |
| C8 beta chain (C8B) | |
| Collagen alpha-2(I) chain | NP_000080 |
| (COL1A2) | |
| Histone H2B type 1-K | NP_542160 |
| (HIST1H2BK) | |
| Plasminogen (PLG) | NP_000292 (Isoform 1) |
| | NP_001161810 (Isoform 2) |
| Phospholipid transfer | NP_006218 |
| protein (PLTP) | |
| Lactotransferrin (LTF) | NP_002334 (Isoform 1) |
| | NP_001186078 (Isoform 2) |
| Vimentin (VIM) | NP_003371 |
| Histone H4 (HIST1H4A) | NP_003529 |
| Apolipoprotein A-IV | NP_000473 |
| (APOA4) | |
| Multimerin-1 (MMRN1) | NP_031377 |
| Clusterin (CLU) | NP_001822 |
| Apolipoprotein C-III | NP_000031 |
| (APOC3) | |
| Vitronectin (VTN) | NP_000629 |
| Endothelial cell-specific | NP_008967 (Isoform a) |
| molecule 1 (ESM1) | NP_001129076 (Isoform b) |
| SPARC (SPARC) | NP_003109 |
| Sushi repeat-containing | NP_006298 |
| protein (SRPX) | |
| Lumican (LUM) | NP_002336 |
| Cation-independent | NP_000867 |
| mannose-6-phosphate | |
| receptor (IGF2R) | |
| Coagulation factor V (F5) | NP_000121 |
| Pentraxin-related protein | NP_002843 |
| PTX3 (PTX3) | |
| Aquaporin 1 | BAA09715 |
| 60S acidic ribosomal | NP_000995 |
| protein P2 | |
| 60S acidic ribosomal | NP_000993 |
| protein P0 | |
| Caveolin-1 | NP_001744 (Isoform alpha) |
| | NP_001166368 (Isoform |
| | beta) |
| Epidermal growth factor | NP_001972 (Isoform A) |
| receptor substrate 15 | NP_001153441 (Isoform B) |
| Cdc42 effector protein 4 | NP_036253 |
| CLIP-associating protein 2 | NP_055912 (Isoform 1) |
| | NP_001193973 Isoform 2) |

1. A method for predicting or diagnosing pulmonary artery hypertension (PAH) in a subject having PAH, at risk of having PAH, or suspected of having PAH, the method comprising:
(a) obtaining a sample from a subject at risk of having PAH;
(b) detecting a level of expression of at least one biomarker in the sample, wherein the at least one biomarker is selected from the group consisting of Protein S100-A8, Protein S100-A9, Alpha-1B-glycoprotein (A1BG), Beta-2-microglobulin (B2M), Calponin-1 (CNN1), Carbonic anhydrase (CA3), (Complement C4-A (C4A), Tenascin-X (TNXB), Pulmonary surfactant-associated protein C (SFTPC), Uteroglobin (SCGB1A1), Periostin (POSTN), Apolipoprotein A-II (APOA2), Collagen alpha-1(XIV) chain (COL14A1), Complement C3 (C3), Apolipoprotein A-1 (APOA1), Antithrombin-III (SERPINC1), von Willebrand factor (VWF), High mobility group protein B1 (HMGB1), Flavin reductase (NADPH) (BLVRB), Fibulin-1 (FBLN1), Heat shock protein beta-6 (HSPB6), BTB/POZ domain-containing protein (KCTD12), Zyxin (ZYX), Carbonic anhydrase 1 (CA1), Alcohol dehydrogenase 1B (ADH1B), Fibulin-5 (FBLN5), Neutrophil gelatinase-associated lipocalin (LCN2), Serpin H1 (SERPINH1), Periaxin (PRX), Protein S100-A12 (S100A12), Myeloblastin (PRTN3), Alpha-2-macroglobulin (A2M), Serotransferrin (TF), Histone H2B type 1 (HIST1H2BK), Isoform 2 of collagen alpha-1(XVIII) chain (COL18A1), Basement membrane-specific heparin sulfate proteoglycan core protein (HSPG2), Fibrillin-1 (FBN1), Bone marrow stromal antigen 2 (BST2), Matrix metalloproteinase-9 (MMP9), Periplakin (PPL), Serum amyloid A-1 (SAM), Thrombospondin-1 (THBS1), Tubulin-specific chaperone A (TBCA), Serine-tRNA ligase, cytoplasmic (SARS), and Aldose reductase (AKR1B1);
(c) comparing the levels of the at least one biomarker to the levels of the at least one biomarker in a control sample from a subject or subjects that do not have PAH; and
wherein a significant difference between the levels of the at least one biomarker in the sample and the levels of the at least one biomarker in the control sample is indicative that subject has or is susceptible to developing PAH.

2. The method of claim 1, further comprising informing the patient or a treating physician of the susceptibility of the patient to PAH.

3. The method of claim 1, wherein the method further comprises a step of administering a therapeutically effective amount of a vasodilator to the subject having PAH.

4. The method of claim 1, wherein the at least one biomarker is selected from the group consisting of Protein S100-A8, Protein S100-A9, and uteroglobulin.

5. The method of claim 1, wherein a combination of at least two biomarkers in the sample is detected.

6. The method of claim 1, wherein the sample is selected from the group consisting of lung tissue, blood, plasma, saliva, urine, and serum.

7. The method of claim 1, wherein detecting the level of expression of the at least one biomarker occurs by using a mass spectrometry method or an immunoassay method.

8. The method of claim 7, wherein the mass spectrometry method comprises selected reaction monitoring (SRM) or multiple reaction monitoring (MRM).

9. The method of claim 1, wherein the pulmonary artery hypertension (PAH) is selected from the group consisting of idiopathic pulmonary artery hypertension (IPAH), associated pulmonary artery hypertension (APAH), PAH caused by structural heart disease, PAH caused by lung disease, PAH caused by inflammatory disease, PAH caused by heart failure, PAH caused by congenital heart disease, and PAH in the newborn.

10. The method of claim 1, wherein a significant difference means at least a 1.5 fold difference between the levels of the at least one biomarker in the sample and the levels of the at least one biomarker in the control sample.

11. A method for predicting or diagnosing hypoxia, hypoxic pulmonary artery hypertension (PAH), normoxic PAH, and no hypoxia or PAH in a subject having hypoxia and/or PAH, at risk of having hypoxia and/or PAH, or suspected of having hypoxia and/or PAH, the method comprising:
(a) obtaining a sample from a subject at risk of having hypoxia and/or PAH;
(b) detecting a level of expression of at least one biomarker in the sample, wherein the at least one biomarker is selected from the group consisting of Mucin-16 (MUC16), Collagen alpha-1(II) chain (COL2A1), Complement factor H (CFH), Complement C1q tumor necrosis factor-related protein 3 (C1QTNF3), Pantetheinase (VNN1), Complement component C8 beta chain (C8B), Collagen alpha-2(I) chain (COL1A2), Histone H2B type 1-K (HIST1H2BK), Plasminogen (PLG), Phospholipid transfer protein (PLTP), Lactotransferrin (LTF), Vimentin (VIM), Histone H4 (HIST1H4A), Apolipoprotein A-IV (APOA4), Multimerin-1 (MMRN1), Clusterin (CLU), Apolipoprotein C-III (APOC3), Vitronectin (VTN), Endothelial cell-specific molecule 1 (ESM1), SPARC, Sushi repeat-containing protein (SRPX), Lumican (LUM), Cation-independent mannose-6-phosphate receptor (IGF2R), Coagulation factor V (F5), Periostin (POSTN), and Pentraxin-related protein PTX3 (PTX3);
(c) comparing the levels of the at least one biomarker in the sample to the levels of the at least one biomarker in a corresponding control sample; and
wherein a significant difference between the levels of the at least one biomarker in the sample and the levels of the at least one biomarker in the control sample is indicative that subject has or is susceptible to developing hypoxia and/or PAH.

12. The method of claim 11, further comprising informing the patient or a treating physician of the susceptibility of the patient to PAH and/or hypoxia.

13. The method of claim 11, wherein the method further comprises a step of administering a therapeutically effective amount of a vasodilator to the subject having PAH.

14. The method of claim 11, wherein the at least one biomarker in the sample is selected from the group consisting of Mucin-16 (MUC16), Collagen alpha-1(II) chain (COL2A1), and Complement factor H (CFH), and wherein the levels of the at least one biomarker change in the subject when the subject is likely to get or has PAH as compared to the levels of biomarker in a subject or subjects that do not have PAH.

15. The method of claim 11, wherein the at least one biomarker in the sample is selected from the group consisting of Complement C1q tumor necrosis factor-related protein 3 (C1QTNF3), Pantetheinase (VNN1), Complement component C8 beta chain (C8B), Collagen alpha-2(I) chain (COL1A2), Histone H2B type 1-K (HIST1H2BK), Plasminogen (PLG), Phospholipid transfer protein (PLTP), and Lactotransferrin (LTF), wherein the levels of the at least one biomarker change in the subject when the subject is likely to get or has PAH without hypoxia as compared to the levels of biomarker in a subject that does not have PAH or hypoxia, and wherein no significant difference is seen if the subject has hypoxic PAH as compared to a subject that has only hypoxia and no PAH.

16. The method of claim 11, wherein the at least one biomarker in the sample is selected from the group consisting of Vimentin (VIM), Histone H4 (HIST1H4A), Apolipoprotein A-IV (APOA4), Multimerin-1 (MMRN1), Clusterin (CLU), and Apolipoprotein C-III (APOC3), wherein the levels of the at least one biomarker change in the subject when the subject is likely to get hypoxic PAH as compared to the levels of biomarker in a subject that only has hypoxia, and wherein no significant difference is seen if the subject has normoxic PAH as compared to a subject that has normoxia and no PAH.

17. The method of claim 11, wherein the at least one biomarker in the sample is selected from the group consisting of Vitronectin (VTN), Endothelial cell-specific molecule 1 (ESM1), and SPARC, and wherein the levels of the at least one biomarker increase in the subject when the subject is likely to get hypoxic PAH as compared to the levels of biomarker in a subject that only has hypoxia and decrease in the subject when the subject is likely to get normoxic PAH as compared to the levels of biomarker in a subject that has no hypoxia or PAH.

18. The method of claim 11, wherein the at least one biomarker in the sample is selected from the group consisting of Sushi repeat-containing protein (SRPX), Lumican (LUM), Cation-independent mannose-6-phosphate receptor (IGF2R), and Coagulation factor V (F5), and wherein the levels of the at least one biomarker decrease in the subject when the subject is likely to get hypoxic PAH as compared to the levels of biomarker in a subject that only has hypoxia and increase in the subject when the subject is likely to get normoxic PAH as compared to the levels of biomarker in a subject that has no hypoxia or PAH.

19. The method of claim 11, wherein detecting the level of expression of the at least one biomarker occurs by using a mass spectrometry method or an immunoassay method.

20. The method of claim 19, wherein the mass spectrometry method comprises selected reaction monitoring (SRM) or multiple reaction monitoring (MRM).

21. The method of claim 11, wherein a combination of at least two biomarkers in the sample is detected.

22. The method of claim 11, wherein the sample is selected from the group consisting of lung tissue, blood, plasma, saliva, urine, and serum.

23. The method of claim 11, wherein a significant difference means at least a 1.5 fold difference between the levels of the at least one biomarker in the sample and the levels of the at least one biomarker in the control sample.

24. A method for predicting or diagnosing pulmonary artery hypertension (PAH) in a subject having PAH, at risk of having PAH, or suspected of having PAH by detecting phosphorylation differences on a protein, the method comprising:
(a) obtaining a sample from a subject at risk of having PAH;
(b) detecting one or more phosphorylation sites on at least one protein in the sample selected from the group consisting of Aquaporin 1, 60S acidic ribosomal protein P2, 60S acidic ribosomal protein PO, Caveolin-1, Epidermal growth factor receptor substrate 15, Cdc42 effector protein 4, and CLIP-associating protein 2;
(c) comparing the phosphorylation sites of the at least one protein in the sample to the phosphorylation sites of the at least one protein in a control sample from a subject or subjects that do not have PAH; and
wherein a phosphorylation difference between at least one protein in the sample and the at least one protein in the control sample is indicative that the subject has or is susceptible to developing PAH.

25. The method of claim 24, further comprising informing the patient or a treating physician of the susceptibility of the patient to PAH.

26. The method of claim 24, wherein the method further comprises a step of administering a therapeutically effective amount of a vasodilator to the subject having PAH.

27. The method of claim 24, wherein a combination of at least two proteins in the sample are detected.

28. The method of claim 24, wherein the sample is a sample selected from the group consisting of lung tissue, blood, plasma, saliva, urine, and serum.

29. The method of claim 24, wherein the pulmonary artery hypertension (PAH) is selected from the group consisting of idiopathic pulmonary artery hypertension (IPAH), associated pulmonary artery hypertension (APAH), PAH caused by structural heart disease, PAH caused by lung disease, PAH caused by inflammatory disease, PAH caused by heart failure, PAH caused by congenital heart disease, and PAH in the newborn.

30. A method for determining the efficacy of vasodilator therapy in a subject undergoing thereof, the method comprising:
(a) obtaining a sample from the subject undergoing vasodilator therapy;
(b) detecting a level of expression of at least one biomarker in the sample, wherein the at least one biomarker is selected from the group consisting of Protein S100-A8, Protein S100-A9, Protein S100-A7, Polyubiquitin-B, Protein S100-A12, Plasma kallikrein, Lymphatic vessel endothelial hyaluronic acid receptor 1, Gamma-glutamyl hydrolase, Tetranectin, Bone-derived growth factor, Platelet basic protein, Insulin-like growth factor-binding protein 3, Pigment epithelium-derived factor, Protein S100-A11 calcium binding protein, Prostaglandin-H2 D-isomerase, Transforming growth factor-beta-induced protein, Vasorin, Kallistatin, Osteopontin, L-selectin, Hepatocyte growth factor activator, and Proliferation-inducing protein 33; and
(c) comparing the levels of the at least one biomarker in the sample from the subject undergoing vasodilator therapy to the levels of the at least one biomarker in a previous sample from the subject, wherein a significant difference in the levels of the at least one biomarker in the sample from the subject undergoing vasodilator therapy as compared to the levels of the at least one biomarker in the previous sample is indicative that the vasodilator therapy is effective.

31. The method of claim 30, further comprising informing the patient or a treating physician of the effectiveness of the vasodilator therapy.

32. The method of claim 30, wherein the method further comprises a step of administering a therapeutically effective amount of a vasodilator to the subject having PAH.

33. The method of claim 30, wherein the at least one biomarker is selected from the group consisting of Protein S100-A8 and Protein S100-A9.

34. The method of claim 30, wherein detecting the level of expression of at least one biomarker occurs by using a mass spectrometry method or an immunoassay method.

35. The method of claim 34, wherein the mass spectrometry method comprises selected reaction monitoring (SRM) or multiple reaction monitoring (MRM).

36. The method of claim 30, wherein a combination of at least two biomarkers in the sample is detected.

37. The method of claim 30, wherein the sample is a selected from the group consisting of lung tissue, blood, plasma, saliva, urine, and serum.

38. The method of claim 30, wherein a significant difference means at least a 1.5 fold change between the change in the levels of the at least one biomarker in the sample from the subject undergoing vasodilator therapy as compared to the levels of the at least one biomarker in the previous sample.

39. A method for screening for a new pulmonary artery hypertension (PAH) therapy, the method comprising:
(a) administering a new therapy to a subject known to have PAH;
(b) obtaining a sample from the subject;
(c) detecting a level of expression of at least one biomarker in the sample, wherein the at least one biomarker is selected from the group consisting of Protein S100-A8, Protein S100-A9, Alpha-1B-glycoprotein (A1 BG), Beta-2-microglobulin (B2M), Calponin-1 (CNN1), Carbonic anhydrase (CA3), (Complement C4-A (C4A), Tenascin-X (TNXB), Pulmonary surfactant-associated protein C (SFTPC), Uteroglobin (SCGB1A1), Periostin (POSTN), Apolipoprotein A-II (APOA2), Collagen alpha-1(XIV) chain (COL14A1), Complement C3 (C3), Apolipoprotein A-1 (APOA1), Antithrombin-III (SERPINC1), von Willebrand factor (VWF), High mobility group protein B1 (HMGB1), Flavin reductase (NADPH) (BLVRB), Fibulin-1 (FBLN1), Heat shock protein beta-6 (HSPB6), BTB/POZ domain-containing protein (KCTD12), Zyxin (ZYX), Carbonic anhydrase 1 (CA1), Alcohol dehydrogenase 1B (ADH1B), Fibulin-5 (FBLN5), Neutrophil gelatinase-associated lipocalin (LCN2), Serpin H1 (SERPINH1), Periaxin (PRX), Protein S100-A12 (S100A12), Myeloblastin (PRTN3), Alpha-2-macroglobulin (A2M), Serotransferrin (TF), Histone H2B type 1 (HIST1H2BK), Isoform 2 of collagen alpha-1(XVIII) chain (COL18A1), Basement membrane-specific heparin sulfate proteoglycan core protein (HSPG2), Fibrillin-1 (FBN1), Bone marrow stromal antigen 2 (BST2), Matrix metalloproteinase-9 (MMP9), Periplakin (PPL), Serum amyloid A-1 (SAM), Thrombospondin-1 (THBS1), Tubulin-specific chaperone A (TBCA), Serine-tRNA ligase, cytoplasmic (SARS), and Aldose reductase (AKR1B1);
(d) comparing the levels of the at least one biomarker to the levels of the at least one biomarker in a control sample from a subject or subjects that do not have PAH or to a previous sample from the subject administered the new therapy; and
wherein a significant difference between the levels of the at least one biomarker in the sample and levels of the at least one biomarker in the control sample or the previous sample from the subject administered the new therapy is indicative that the new PAH therapy is effective.

40. The method of claim 39, wherein the new therapy is a drug.

41. The method of claim 39, wherein the subject is a human or an animal.

42. The method of claim 39, wherein a significant difference means at least a 1.5 fold difference between the levels of the at least one biomarker in the sample and the levels of the at least one biomarker in the control sample or the previous sample from the subject.

43. A diagnostic kit for determining predicting or diagnosing pulmonary artery hypertension (PAH) in a subject having PAH, at risk of having PAH, or suspected of having PAH, the kit comprising:
(a) a substrate for collecting a biological sample from the patient; and
(b) means for measuring the levels of one or more biomarkers selected from the group consisting of Protein S100-A8, Protein S100-A9, Alpha-1B-glycoprotein (A1BG), Beta-2-microglobulin (B2M), Calponin-1 (CNN1), Carbonic anhydrase (CA3), (Complement C4-A (C4A), Tenascin-X (TNXB), Pulmonary surfactant-associated protein C (SFTPC), Uteroglobin (SCGB1A1), Periostin (POSTN), Apolipoprotein A-II (APOA2), Collagen alpha-1(XIV) chain (COL14A1), Complement C3 (C3), Apolipoprotein A-1 (APOA1), Antithrombin-III (SERPINC1), von Willebrand factor (VWF), High mobility group protein B1 (HMGB1), Flavin reductase (NADPH) (BLVRB), Fibulin-1 (FBLN1), Heat shock protein beta-6 (HSPB6), BTB/POZ domain-containing protein (KCTD12), Zyxin (ZYX), Carbonic anhydrase 1 (CA1), Alcohol dehydrogenase 1B (ADH1B), Fibulin-5 (FBLN5), Neutrophil gelatinase-associated lipocalin (LCN2), Serpin H1 (SERPINH1), Periaxin (PRX), Protein S100-A12 (S100A12), Myeloblastin (PRTN3), Alpha-2-macroglobulin (A2M), Serotransferrin (TF), Histone H2B type 1 (HIST1H2BK), Isoform 2 of collagen alpha-1(XVIII) chain (COL18A1), Basement membrane-specific heparin sulfate proteoglycan core protein (HSPG2), Fibrillin-1 (FBN1), Bone marrow stromal antigen 2 (BST2), Matrix metalloproteinase-9 (MMP9), Periplakin (PPL), Serum amyloid A-1 (SAA1), Thrombospondin-1 (THBS1), Tubulin-specific chaperone A (TBCA), Serine-tRNA ligase, cytoplasmic (SARS), and Aldose reductase (AKR1B1).

44. A diagnostic kit for predicting or diagnosing hypoxia, hypoxic pulmonary artery hypertension (PAH), normoxic PAH, and no hypoxia or PAH in a subject having hypoxia and/or PAH, at risk of having hypoxia and/or PAH, or suspected of having hypoxia and/or PAH, the kit comprising:
(a) a substrate for collecting a biological sample from the patient; and
(b) means for measuring the levels of one or more biomarkers selected from the group consisting of Mucin-16 (MUC16), Collagen alpha-1(II) chain (COL2A1), Complement factor H (CFH), Complement C1q tumor necrosis factor-related protein 3 (C1QTNF3), Pantetheinase (VNN1), Complement component C8 beta chain (C8B), Collagen alpha-2(I) chain (COL1A2), Histone H2B type 1-K (HIST1H2BK), Plasminogen (PLG), Phospholipid transfer protein (PLTP), Lactotransferrin (LTF), Vimentin (VIM), Histone H4 (HIST1H4A), Apolipoprotein A-IV (APOA4), Multimerin-1 (MMRN1), Clusterin (CLU), Apolipoprotein C-III (APOC3), Vitronectin (VTN), Endothelial cell-specific molecule 1 (ESM1), SPARC, Sushi repeat-containing protein (SRPX), Lumican (LUM), Cation-independent mannose-6-phosphate receptor (IGF2R), Coagulation factor V (F5), Periostin (POSTN), and Pentraxin-related protein PTX3 (PTX3).

45. A diagnostic kit for predicting or diagnosing pulmonary artery hypertension (PAH) in a subject having PAH, at risk of having PAH, or suspected of having PAH by detecting phosphorylation differences on a protein, the kit comprising:
(a) a substrate for collecting a biological sample from the patient; and
(b) means for measuring the levels of one or more biomarkers selected from the group consisting of Aquaporin 1, 60S acidic ribosomal protein P2, 60S acidic ribosomal protein PO, Caveolin-1, Epidermal growth factor receptor substrate 15, Cdc42 effector protein 4, and CLIP-associating protein 2.

46. A diagnostic kit for determining the efficacy of vasodilator therapy in a subject undergoing thereof, the kit comprising:
(a) a substrate for collecting a biological sample from the patient; and
(b) means for measuring the levels of one or more biomarkers selected from the group consisting of Protein S100-A8, Protein S100-A9, Protein S100-A7, Polyubiquitin-B, Protein S100-A12, Plasma kallikrein, Lymphatic vessel endothelial hyaluronic acid receptor 1, Gamma-glutamyl hydrolase, Tetranectin, Bone-derived growth factor, Platelet basic protein, Insulin-like growth factor-binding protein 3, Pigment epithelium-derived factor, Protein S100-A11 calcium binding protein, Prostaglandin-H2 D-isomerase, Transforming growth factor-beta-induced protein, Vasorin, Kallistatin, Osteopontin, L-selectin, Hepatocyte growth factor activator, and Proliferation-inducing protein 33.
